(19) United States
(12) Patent Application Publication
(10) Pub. No.: US 2023/0146719 A1
(43) Pub. Date: May 11, 2023

(54) CRYSTALLINE N-METHYL TRYPTAMINE DERIVATIVES

(71) Applicant: CAAMTECH, INC., Issaquah, WA (US)

(72) Inventor: Andrew R. CHADEAYNE, Issaquah, WA (US)

(73) Assignee: CAAMTECH, INC., Issaquah, WA (US)

(21) Appl. No.: 17/987,208

(22) Filed: Nov. 15, 2022

Related U.S. Application Data
(63) Continuation-in-part of application No. 17/722,914, filed on Apr. 18, 2022, now abandoned.
Application No. 17/722,914 is a continuation of application No. 16/827,072, filed on Mar. 23, 2020, now Pat. No. 11,332,441.

(51) Int. Cl. C07D 209/16

(52) U.S. Cl. CPC C07D 209/16; C07B 2200/13

(57) ABSTRACT

Crystalline N-methyl tryptamine derivatives, compositions containing those crystalline forms and their methods of use are disclosed. The crystalline N-methyl tryptamine derivatives according to the invention include crystalline N-methyl-N-propyltryptamine (MPT), crystalline N-methyl-N-isopropyltryptammonium fumarate (MiPT fumarate), crystalline 4-hydroxy-N-methyl-N-isopropyltryptammonium fumarate monohydrate (HO-MiPT fumarate monohydrate), and crystalline [2-(1H-indol-3-yl)ethyl]trimethylazanium iodide (N,N,N-trimethyltryptammonium iodide or TMT iodide), such as crystalline form 1 of TMT iodide.

### CROSS-REFERENCE TO RELATED APPLICATIONS

This application is a Continuation-in-Part Application of U.S. application Ser. No. 17/722,914, filed on Apr. 18, 2022; which is a Continuation Application of U.S. application Ser. No. 16/827,072, filed on Mar. 23, 2020, which granted as U.S. Pat. No. 11,332,441 on May 17, 2022; each of which is hereby incorporated by reference.

### FIELD OF THE INVENTION

This invention relates to crystalline N-methyl tryptamine derivatives, compositions containing those crystalline forms and their methods of using the same. The crystalline N-methyl tryptamine derivatives according to the disclosure include crystalline N-methyl-N-propyltryptamine (MPT), crystalline N-methyl-N-isopropyltryptammonium fumarate (MiPT fumarate), crystalline 4-hydroxy-N-methyl-N-isopropyltryptammonium fumarate monohydrate (HO-MiPT fumarate monohydrate), and/or crystalline [2-(1H-indol-3-yl)ethyl]trimethylazanium iodide (N,N,N-trimethyltryptammonium iodide or TMT iodide), such as crystalline form 1 of TMT iodide.

### BACKGROUND OF THE INVENTION

N-methyl-N-propyltryptamine (MPT) is a structural analog of N,N-dimethyltryptamine (DMT), which is a well-known ‘psychedelic’ molecule found in a variety of naturally occurring organisms, including plants, animals, and fungi, including mushrooms. In humans, DMT is the only known endogenous mammalian N,N-dimethylated trace amine (Fontanilla et al., 2009). Naturally occurring tryptamines (e.g. DMT, psilocybin, 5-methoxy-N,N-dimethyltryptamine) and their synthetic derivatives (e.g. psilacetin, MPT) have garnered considerable attention of late due to new evidence demonstrating their efficacy in treating mood (e.g. anxiety and depression) and post-traumatic stress disorders (PTSDs) (Aixala' et al., 2018; Cameron et al., 2019).

Psilocybin, isolated from the so-called “magic” mushrooms, is perhaps the best-known prodrug of the serotonin 2a agonist psilocin (Nichols, 2016). Recent studies indicate that psilocin (and its prodrugs like psilocybin and psilacetin) could provide effective treatment for mood disorders, end-of-life anxiety, addiction, and PTSD (Carhart-Harris et al., 2016; Johnson & Griffiths, 2017). However, the long duration of action of psilocin and its prodrugs can result in practical challenges for both patients and clinicians (Passie et al., 2002). Accordingly, the mental health industry would benefit from exploring alternative tryptamine treatment options that provide similar therapeutic benefits while having a shorter duration of action.

While the synthesis of DMT was first reported in 1931 (Manske, 1931), the first literature report of MPT appeared in 2005 (Brandt et al., 2005) and it has not undergone significant study. Prior to this work, however, no crystalline form of MPT has been reported. There is a need therefore to develop a form of MPT that allows for development as an active pharmaceutical ingredient (an API) and for pharmaceutical compositions containing that form of MPT. This disclosure answers such needs.

Although therapeutic efficacy is the primary concern for an active pharmaceutical ingredient (API), the salt and solid state form (i.e., the crystalline or amorphous form) of a drug candidate can be critical to its pharmacological properties, such as bioavailability, and to its development as a viable API. Recently, crystalline forms of API's have been used to alter the physicochemical properties of an API. Each crystalline form of a drug candidate can have different solid state (physical and chemical) properties. The differences in physical properties exhibited by a novel solid form of an API (such as a cocrystal or polymorph of the original therapeutic compound) affect pharmaceutical parameters such as storage stability, compressibility and density (important in formulation and product manufacturing), and solubility and dissolution rates (important factors in determining bioavailability). Because these practical physical properties are influenced by the solid-state properties of the crystalline form of the API, they can significantly impact the selection of a compound as an API, the ultimate pharmaceutical dosage form, the optimization of manufacturing processes, and absorption in the body. Moreover, finding the most adequate solid-state form for further drug development can reduce the time and the cost of that development.

Obtaining crystalline forms of an API is extremely useful in drug development. It permits better characterization of the drug candidate's chemical and physical properties. Crystalline forms often have better chemical and physical properties than the API in its amorphous state. Such crystalline forms may possess more favorable pharmaceutical and pharmacological properties or be easier to process.

### SUMMARY OF THE DISCLOSURE

This disclosure relates to new crystalline forms of N-methyl tryptamine derivatives, specifically crystalline N-methyl-N-propyltryptamine (MPT), crystalline N-methyl-N-isopropyltryptammonium fumarate (MiPT fumarate), crystalline 4-hydroxy-N-methyl-N-isopropyltryptammonium fumarate monohydrate (HO-MiPT fumarate monohydrate), or crystalline [2-(1H-indol-3-yl)ethyl]trimethylazanium iodide (N,N,N-trimethyltryptammonium iodide or TMT iodide), such as crystalline form 1 of TMT iodide (collectively, the “crystalline N-methyl tryptamine derivatives according to the disclosure”).

In one embodiment, crystalline N-methyl-N-propyltryptamine (MPT) according to the disclosure is characterized by an orthorhombic, Pbca crystal system space group at a temperature of about 200 K; unit cell dimensions α=13.5715 (11) Å, b=12.4352(10) Å, c=15.1627(12) Å, at a temperature of about 200 K; an x-ray powder diffraction (XRPD) pattern substantially similar to FIG. 3; or an x-ray powder diffraction (XRPD) pattern having peaks at 11.7, 13.4 and 19.4° 20±0.2° 20.

In one embodiment, crystalline N-methyl-N-isopropyltryptammonium fumarate (MiPT fumarate) according to the disclosure is characterized by a monoclinic, P2/c crystal system space group at a temperature of about 200 K; unit cell dimensions α=9.852 (2) Å, b=12.789(2) Å, c=14.875 (3) Å, and β=106.932 (7°) at a temperature of about 200 K; an x-ray powder diffraction pattern substantially similar to FIG. 7; or an x-ray powder diffraction (XRPD) pattern having peaks at 11.9, 14.9 and 16.9° 2θ±0.2° 2θ.

In one embodiment, crystalline 4-hydroxy-N-methyl-N-isopropyltryptammonium fumarate monohydrate (HO-MiPT fumarate monohydrate) according to the disclosure is characterized by a monoclinic, C2/c crystal system space group at a temperature of about 200 K; unit cell dimensions α=29.507 (3) Å, b=8.7445(8) Å, c=17.3659 (18) Å, and β=123.389 (18)° at a temperature of about 200 K; an x-ray powder diffraction pattern substantially similar to FIG. 11; or an x-ray powder diffraction (XRPD) pattern having peaks at 7.2, 13.4 and 18.0° 20±0.2° 2θ.

In one embodiment, this disclosure pertains to particular crystalline forms of TMT iodide, including crystalline form 1 of TMT iodide. In one embodiment, crystalline form 1 of TMT iodide is characterized by at least one of: a orthorhombic, P2₁2₁2₁ space group at a temperature of about 297(2) K; unit cell dimensions a=9.4322(6) Å, b=11.4021(7) Å, c=13.3328(7) Å, α=900, β=900, and γ=90°; an X-ray powder diffraction (XRPD) pattern substantially similar to FIG. 14; and an X-ray powder diffraction pattern characterized by at least two peaks selected from 10.2, 13.9, 16.9. and 21.4° 2θ±0.2° 2θ.

The disclosure also relates to compositions comprising a crystalline N-methyl tryptamine derivative according to the disclosure, specifically crystalline N-methyl-N-propyltryptamine (MPT), crystalline N-methyl-N-isopropyltryptammonium fumarate (MiPT fumarate), crystalline 4-hydroxy-N-methyl-N-isopropyltryptammonium fumarate monohydrate (HO-MiPT fumarate monohydrate), or crystalline N,N,N-trimethyltryptammonium iodide (TMT iodide), such as crystalline form 1 of TMT iodide, and an excipient.

The disclosure also relates to compositions comprising a combination of, as a first component, a crystalline N-methyl tryptamine derivative according to the disclosure, specifically crystalline N-methyl-N-propyltryptamine (MPT), crystalline N-methyl-N-isopropyltryptammonium fumarate (MiPT fumarate), crystalline 4-hydroxy-N-methyl-N-isopropyltryptammonium fumarate monohydrate (HO-MiPT fumarate monohydrate), or crystalline N,N,N-trimethyltryptammonium iodide (TMT iodide), such as crystalline form 1 of TMT iodide, and a second component selected from at least one of (a) a serotonergic drug, (b) a purified psilocybin derivative, (c) a purified cannabinoid, (d) a purified terpene, (e) an adrenergic drug, (f) a dopaminergic drug, (g) a monoamine oxidase inhibitor, (h) a purified erinacine, and (i) a purified hericenone; and at least one excipient.

The disclosure also relates to a method of preventing or treating a physical and/or psychological disorder comprising the step of administering to a subject in need thereof an effective amount of a crystalline N-methyl tryptamine derivative according to the disclosure, specifically crystalline N-methyl-N-propyltryptamine (MPT), crystalline N-methyl-N-isopropyltryptammonium fumarate (MiPT fumarate), crystalline 4-hydroxy-N-methyl-N-isopropyltryptammonium fumarate monohydrate (HO-MiPT fumarate monohydrate), or crystalline N,N,N-trimethyltryptammonium iodide (TMT iodide), such as crystalline form 1 of TMT iodide, or a composition according to the disclosure.

The disclosure further relates to a method of preventing or treating inflammation and/or pain, preventing or treating a neurological disorder, modulating activity of a mitogen-activated protein kinase (MAPK), modulating neurogenesis, or modulating neurite outgrowth comprising the step of administering to a subject in need thereof an effective amount of a crystalline N-methyl tryptamine derivative according to the disclosure, specifically crystalline N-methyl-N-propyltryptamine (MPT), crystalline N-methyl-N-isopropyltryptammonium fumarate (MiPT fumarate), crystalline 4-hydroxy-N-methyl-N-isopropyltryptammonium fumarate monohydrate (HO-MiPT fumarate monohydrate), or crystalline N,N,N-trimethyltryptammonium iodide (TMT iodide), such as crystalline form 1 of TMT iodide, or a composition according to the disclosure.

As used herein, the term “a subject in need thereof” refers to a person requiring a composition to treat a particular disease or condition (e.g., inflammation, pain, a psychological disorder, modulating activity at a receptor, etc.). In one embodiment, the “subject in need thereof” may be identified by analyzing, diagnosing, and/or determining whether the person (or subject) requires the composition for treatment of a particular disease or condition. In one embodiment, identifying a person in need of treatment comprises diagnosing a person with a medical condition, e.g., a neurological disorder, a chemical imbalance, a hereditary condition, etc. In one embodiment, identifying a person in need of treatment comprises performing a psychiatric evaluation. In one embodiment, identifying a person in need of treatment comprises performing a blood test. In one embodiment, identifying a person in need of treatment comprises determining whether a person has a compulsive disorder. In one embodiment, identifying a person in need of treatment comprises self-identifying as having a compulsive disorder.

### BRIEF DESCRIPTION OF FIGURES

FIG. 1 depicts the molecular structure of crystalline MPT according to the disclosure with atomic labelling.

FIG. 2 shows the crystal packing of crystalline MPT, viewed along the c axis.

FIG. 3 is a simulated X-ray powder diffraction pattern (XRPD) for crystalline MPT generated from its single crystal data.

FIG. 4 shows the molecular structure of crystalline MiPT fumarate with atomic labelling.

FIG. 5 shows the hydrogen bonding of the fumarate ion in the structure of crystalline MiPT fumarate.

FIG. 6 shows the crystal packing of crystalline MiPT fumarate, viewed along the axis.

FIG. 7 is a simulated X-ray powder diffraction pattern (XRPD) for crystalline MiPT fumarate generated from its single crystal data.

FIG. 8 shows the molecular structure of crystalline HO-MiPT fumarate monohydrate with atomic labelling.

FIG. 9 shows the hydrogen bonding of the fumarate ion in the structure of crystalline MiPT fumarate.

FIG. 10 shows the crystal packing of crystalline HO-MiPT fumarate monohydrate, viewed along the b axis.

FIG. 11 is a simulated X-ray powder diffraction pattern (XRPD) for HO-MiPT fumarate monohydrate generated from its single crystal data.

FIG. 12 depicts the molecular structure of crystalline form 1 of TMT iodide according to the disclosure with atomic labelling.

FIG. 13 shows the unit cell of crystalline form 1 of TMT iodide along the a-axis.

FIG. 14 shows the simulated X-ray powder diffraction pattern (XRPD) for crystalline form 1 of TMT iodide.

### DETAILED DESCRIPTION

This disclosure relates to crystalline N-methyl tryptamine derivatives, specifically crystalline N-methyl-N-propyltryptamine (MPT), crystalline N-methyl-N-isopropyltryptammonium fumarate (MiPT fumarate), crystalline 4-hydroxy-N-methyl-N-isopropyltryptammonium fumarate monohydrate (HO-MiPT fumarate monohydrate), or crystalline N,N,N-trimethyltryptammonium iodide (TMT iodide), such as crystalline form 1 of TMT iodide (collectively, the “crystalline N-methyl tryptamine derivatives according to the disclosure”), and to pharmaceutical compositions containing a crystalline N-methyl tryptamine derivative according to the disclosure. The therapeutic uses of the crystalline N-methyl tryptamine derivatives according to the disclosure, are described below as well as compositions containing them. The crystalline N-methyl tryptamine derivatives according to the disclosure, and the methods used to characterize it are described in the examples below.

TMT iodide has the following chemical formula:

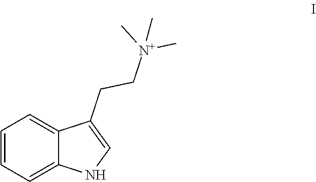

Methods of Treatment and Therapeutic Uses

The crystalline N-methyl tryptamine derivatives according to the disclosure, specifically crystalline N-methyl-N-propyltryptamine (MPT), crystalline N-methyl-N-isopropyltryptammonium fumarate (MiPT fumarate), crystalline 4-hydroxy-N-methyl-N-isopropyltryptammonium fumarate monohydrate (HO-MiPT fumarate monohydrate), or crystalline N,N,N-trimethyltryptammonium iodide (TMT iodide), such as crystalline form 1 of TMT iodide, the methods and the pharmaceutical compositions of the disclosure are used to regulate the activity of a neurotransmitter receptor by administering a therapeutically effective dose of a crystalline N-methyl tryptamine derivative according to the disclosure. In another embodiment, the crystalline N-methyl tryptamine derivatives according to the disclosure, specifically crystalline N-methyl-N-propyltryptamine (MPT), crystalline N-methyl-N-isopropyltryptammonium fumarate (MiPT fumarate), crystalline 4-hydroxy-N-methyl-N-isopropyltryptammonium fumarate monohydrate (HO-MiPT fumarate monohydrate), or crystalline N,N,N-trimethyltryptammonium iodide (TMT iodide), such as crystalline form 1 of TMT iodide, the methods and the pharmaceutical compositions of the disclosure are used to treat inflammation and/or pain by administering a therapeutically effective dose of a crystalline N-methyl tryptamine derivative according to the disclosure. Methods of the disclosure administer a therapeutically effective amount of a crystalline N-methyl tryptamine derivative according to the disclosure to prevent or treat a disease or condition such as those discussed below for a subject in need of treatment. A crystalline N-methyl tryptamine derivative according to the disclosure may be administered neat or as a composition comprising a crystalline N-methyl tryptamine derivative according to the disclosure as discussed below.

A crystalline N-methyl tryptamine derivative according to the disclosure may be used to prevent and/or treat a psychological disorder. The disclosure provides a method for preventing and/or treating a psychological disorder by administering to a subject in need thereof a therapeutically effective amount of a crystalline N-methyl tryptamine derivative according to the disclosure, including the preferred embodiments discussed above. The psychological disorder may be chosen from depression; psychotic disorder; schizophrenia; schizophreniform disorder (acute schizophrenic episode); schizoaffective disorder; bipolar I disorder (mania, manic disorder, manic-depressive psychosis); bipolar II disorder; major depressive disorder; major depressive disorder with psychotic feature (psychotic depression); delusional disorders (paranoia); Shared Psychotic Disorder (Shared paranoia disorder); Brief Psychotic disorder (Other and Unspecified Reactive Psychosis); Psychotic disorder not otherwise specified (Unspecified Psychosis); paranoid personality disorder; schizoid personality disorder; schizotypal personality disorder; anxiety disorder; social anxiety disorder; substance-induced anxiety disorder; selective mutism; panic disorder; panic attacks; agoraphobia; attention deficit syndrome; post-traumatic stress disorder (PTSD), premenstrual dysphoric disorder (PMDD), and premenstrual syndrome (PMS).

A crystalline N-methyl tryptamine derivative according to the disclosure may be used to prevent and/or treat of a brain disorder. The disclosure provides a method for preventing and/or treating a brain disorder by administering to a subject in need thereof a therapeutically effective amount of a crystalline N-methyl tryptamine derivative according to the disclosure, including the preferred embodiments discussed above. The brain disorder is chosen from Huntington's disease, Alzheimer's disease, dementia, and Parkinson's disease.

A crystalline N-methyl tryptamine derivative according to the disclosure may be used to prevent and/or treat developmental disorders, delirium, dementia, amnestic disorders and other cognitive disorders, psychiatric disorders due to a somatic condition, drug-related disorders, schizophrenia and other psychotic disorders, mood disorders, anxiety disorders, somatoform disorders, factitious disorders, dissociative disorders, eating disorders, sleep disorders, impulse control disorders, adjustment disorders, or personality disorders. The disclosure provides a method for preventing and/or treating these disorders by administering to a subject in need thereof a therapeutically effective amount of a crystalline N-methyl tryptamine derivative according to the disclosure, including the preferred embodiments discussed above.

A crystalline N-methyl tryptamine derivative according to the disclosure may be used to prevent and/or treat inflammation and/or pain, such as for example inflammation and/or pain associated with inflamatory skeletal or muscular diseases or conditions. The disclosure provides a method for preventing and/or treating inflammation and/or pain by administering to a subject in need thereof a therapeutically effective amount of a crystalline N-methyl tryptamine derivative according to the disclosure, including the preferred embodiments discussed herein. Generally speaking, treatable “pain” includes nociceptive, neuropathic, and mix-type. A method of the disclosure may reduce or alleviate the symptoms associated with inflammation, including but not limited to treating localized manifestation of inflammation characterized by acute or chronic swelling, pain, redness, increased temperature, or loss of function in some cases. A method of the disclosure may reduce or alleviate the symptoms of pain regardless of the cause of the pain, including but not limited to reducing pain of varying severity, i.e., mild, moderate and severe pain, acute pain and chronic pain. A method of the disclosure is effective in treating joint pain, muscle pain, tendon pain, burn pain, and pain caused by inflammation such as rheumatoid arthritis. Skeletal or muscular diseases or conditions which may be treated include but are not limited to musculoskeletal sprains, musculoskeletal strains, tendonopathy, peripheral radiculopathy, osteoarthritis, joint degenerative disease, polymyalgia rheumatica, juvenile arthritis, gout, ankylosing spondylitis, psoriatic arthritis, systemic lupus erythematosus, costochondritis, tendonitis, bursitis, such as the common lateral epicondylitis (tennis elbow), medial epicondylitis (pitchers elbow) and trochanteric bursitis, temporomandibular joint syndrome, and fibromyalgia.

A crystalline N-methyl tryptamine derivative according to the disclosure may be used to modulate activity of a mitogen-activated protein kinase (MAPK), comprising administering a composition of the invention. MAPKs provide a wide-ranging signaling cascade that allow cells to quickly respond to biotic and abiotic stimuli. Exemplary MAPKs include, but are not limited to, Tropomyosin Receptor Kinase A (TrkA), P38-alpha, and c-Jun N-Terminal Kinase 3 (JNK3). TrkA is a high affinity catalytic receptor of nerve growth factor (NGF) protein. TrkA regulates NGF response, influencing neuronal differentiation and outgrowth as well as programmed cell death. p38-alpha is involved with the regulation of pro-inflammatory cytokines, including TNF-α. In the central nervous system, p38-alpha regulates neuronal death and neurite degeneration, and it is a common target of Alzheimer's disease therapies. JNK3 is a neuronal-specific protein isoform of the JNKs. It is involved with the regulation of apoptosis. JNK3 also plays a role in modulating the response of cytokines, growth factors, and oxidative stress.

As used herein, the term “modulating activity of a mitogen-activated protein kinase” refers to changing, manipulating, and/or adjusting the activity of a mitogen activating protein. In one embodiment, modulating the activity of a MAPK can influence neural health, neurogenesis, neural growth and differentiation, and neurodegenerative diseases.

A crystalline N-methyl tryptamine derivative according to the disclosure may be used to modulate neurogenesis, comprising administering a composition of the invention. As used herein, the term “modulating neurogenesis” refers to changing, manipulating, and/or adjusting the growth and development of neural tissue. In one embodiment, neurogenesis comprises adult neurogenesis, in which new neural stem cells are generated from neural stem cells in an adult animal. In one embodiment, modulating neurogenesis comprises increasing and/or enhancing the rate at which new neural tissue is developed.

A crystalline N-methyl tryptamine derivative according to the disclosure may be used to modulate neurite outgrowth, comprising administering a composition of the invention. As used herein, the term “modulating neurite outgrowth” refers to changing, manipulating, and/or adjusting the growth and development of neural projections, or “neurites.” In one embodiment, neurogenesis comprises modulating the growth of new neurites, the number of neurites per neuron, and/or neurite length. In one embodiment, modulating neurite outgrowth comprises increasing and/or enhancing the rate and/or length at which neurites develop.

A crystalline N-methyl tryptamine derivative according to the disclosure may be used to prevent and/or treat sexual health disorders including, but not limited to, hypoactive sexual desire disorder, hyperactive sexual desire disorder, orgasmic disorder, arousal disorder, vaginismus, and dyspareunia. In some embodiments, the disorder is a male sexual dysfunction disorder. In some embodiments, the disorder is a female sexual dysfunction disorder.

A crystalline N-methyl tryptamine derivative according to the disclosure may be used to prevent and/or treat women's health disorders including, but not limited to, menstrual cramping, dysmenorrhea, post-hysterectomy pain, vaginal or vulvar vestibule mucosa disorder, menopausal-related disorders, vaginal atrophy, or vulvar vestibulitis.

Compositions

The disclosure also relates to compositions comprising an effective amount of a crystalline N-methyl tryptamine derivative according to the disclosure, specifically crystalline N-methyl-N-propyltryptamine (MPT), crystalline N-methyl-N-isopropyltryptammonium fumarate (MiPT fumarate), crystalline 4-hydroxy-N-methyl-N-isopropyltryptammonium fumarate monohydrate (HO-MiPT fumarate monohydrate), or crystalline N,N,N-trimethyltryptammonium iodide (TMT iodide), such as crystalline form 1 of TMT iodide, especially pharmaceutical compositions comprising a therapeutically effective amount of a crystalline N-methyl tryptamine derivative according to the disclosure and a pharmaceutically acceptable carrier (also known as a pharmaceutically acceptable excipient). As discussed above, a crystalline N-methyl tryptamine derivative according to the disclosure may be, for example, therapeutically useful to prevent and/or treat the psychological and other disorders discussed above.

A composition or a pharmaceutical composition of the disclosure may be in any form which contains a crystalline N-methyl tryptamine derivative according to the disclosure. The composition may be, for example, a tablet, capsule, liquid suspension, injectable, topical, or transdermal. The compositions or pharmaceutical compositions generally contain, for example, about 1% to about 99% by weight of a crystalline N-methyl tryptamine derivative according to the disclosure and, for example, 99% to 1% by weight of at least one suitable pharmaceutical excipient. In one embodiment, the composition may be between about 5% and about 75% by weight of a crystalline N-methyl tryptamine derivative according to the disclosure with the rest being at least one suitable pharmaceutical excipient or at least one other adjuvant, as discussed below.

Published US applications US 2018/0221396 A1 and US 2019/0142851 A1 disclose compositions comprising a combination of a first purified psilocybin derivative with a second purified psilocybin derivative, with one or two purified cannabinoids or with a purified terpene. Various ratios of these components in the composition are also disclosed. The disclosures of US 2018/0221396 A1 and US 2019/0142851 A1 are incorporated herein by reference. According to this disclosure, crystalline N-methyl tryptamine derivatives according to the disclosure, specifically crystalline N-methyl-N-propyltryptamine (MPT), crystalline N-methyl-N-isopropyltryptammonium fumarate (MiPT fumarate), crystalline 4-hydroxy-N-methyl-N-isopropyltryptammonium fumarate monohydrate (HO-MiPT fumarate monohydrate), or crystalline N,N,N-trimethyltryptammonium iodide (TMT iodide), such as crystalline form 1 of TMT iodide, may be used as the “first purified psilocybin derivative” in the compositions described in US 2018/0221396 A1 and US 2019/0142851 A1. Accordingly, this disclosure provides a composition comprising: a first component comprising a crystalline N-methyl tryptamine derivative according to the disclosure; at least one second component selected from at least one of (a) a serotonergic drug, (b) a purified psilocybin derivative, (c) a purified cannabinoid, and (d) a purified terpene; and at least one pharmaceutically-acceptable excipient or at least one other adjuvant. Such a composition may be a pharmaceutical composition wherein the components are present individually in therapeutically effective amounts or by combination in a therapeutically effective amount to treat a disease, disorder, or condition as described herein.

When used in such compositions as a first component comprising a crystalline N-methyl tryptamine derivative according to the disclosure with a second component selected from at least one of (a) a serotonergic drug, (b) a purified psilocybin derivative, (c) a purified cannabinoid, and (d) a purified terpene, the compositions represent particular embodiments of the invention. Compositions having as a first component a crystalline N-methyl tryptamine derivative according to the disclosure with a second component selected from at least one of (e) an adrenergic drug, (f) a dopaminergic drug, (g) a monoamine oxidase inhibitor, (h) a purified erinacine, and (i) a purified hericenone represent additional particular embodiments of the invention represented by the compositions having a crystalline N-methyl tryptamine derivative according to the disclosure. In some embodiments, the first and second components can be administered at the same time (e.g., together in the same composition), or at separate times over the course of treating a patient in need thereof. Such a composition may be a pharmaceutical composition wherein the components are present individually in therapeutically effective amounts or by combination in a therapeutically effective amount to treat a disease, disorder, or condition as described herein.

Within the context of this disclosure, the term “purified” means separated from other materials, such as plant or fungal material, e.g., protein, chitin, cellulose, or water. In one embodiment, the term “purified” refers to a compound substantially free of other materials. In one embodiment, the term “purified” refers to a compound that is substantially free from a second tryptamine compound. In one embodiment, the term “purified” refers to a compound substantially free from histidine. In one embodiment, the term “purified” refers to a compound substantially free from a biological material, such as mold, fungus, plant matter, or bacteria. In one embodiment, the term “purified” refers to a compound substantially free from a paralytic.

In one embodiment, the term “purified” refers to a compound which has been separated from other compounds that are typically co-extracted when the purified compound is extracted from a naturally occurring organism. In one embodiment, a “purified” psilocybin derivative is partially or completely isolated from other psilocybin derivatives present in a source material, such as a psilocybin-containing mushroom. In one example, “purified” baeocystin is substantially free from psilocybin and/or psilocin. By contrast, traditional psilocybin mushroom extracts (aka crude extracts or fruit body extracts) would be expected to contain an unpredictable and varying amount of psilocybin, psilocin, baeocystin, norbaeocystin, salts thereof, or combinations thereof. Other examples of unpurified psilocybin derivatives would include mycelium containing psilocybin derivatives and/or naturally occurring fungal material such as biological material and/or structural material such as chitin. Similarly, the term “cannabis extracts” or “cannabinoid extracts” traditionally refers to whole plants (aka crude or full spectrum extracts) which have not been subjected to further purification to eliminate unwanted molecules that naturally occur in the cannabis plant. For example, a “cannabis extract comprising cannabidiol” could be expected to include cannabidiol (aka “CBD”) and also varying amounts of other compounds, including cannabinoids, terpenes, and other biological material.

In one embodiment, the term “purified” refers to a compound or composition that has been crystallized.

In one embodiment, the term “purified” refers to a compound or composition that has been chromatographed, for example by gas chromatography, liquid chromatography (e.g., LC, HPLC, etc.), etc.

In one embodiment, the term “purified” refers to a compound or composition that has been distilled.

In one embodiment, the term “purified” refers to a compound or composition that has been sublimed.

In one embodiment, the term “purified” refers to a compound or composition that has been subject to two or more steps chosen from crystallization, chromatography, distillation, or sublimation.

In one embodiment, the term “purified” refers to a compound that is between 80-100% pure.

In one embodiment, the term “purified” refers to a compound that is between 90-100% pure.

In one embodiment, the term “purified” refers to a compound that is between 95-100% pure.

In one embodiment, the term “purified” refers to a compound that is between 99-100% pure.

In one embodiment, the term “purified” refers to a compound that is between 99.9-100% pure.

A serotonergic drug refers to a compound that binds to, blocks, or otherwise influences (e.g., via an allosteric reaction) activity at a serotonin receptor as described in paragraphs [0245]-[0253] of US 2018/0221396 A1 and [0305]-[0311] US 2019/0142851 A1 as well as the disclosed exemplary embodiments. Exemplary psilocybin derivatives include but are not limited to psilocybin itself and the psilocybin derivatives described in paragraphs [0081]-[0109] of US 2018/0221396 A1 and [0082]-[0110] US 2019/0142851 A1 as well as the disclosed exemplary embodiments. Exemplary cannabinoids include but are not limited to the cannabinoids described in paragraphs [0111]-[0145] of US 2018/0221396 A1 and [0112]-[0146] US 2019/0142851 A1 as well as the disclosed exemplary embodiments. Exemplary terpenes include but are not limited to the terpenes described in paragraphs [0160]-[0238] of US 2018/0221396 A1 and [0161]-[0300] US 2019/0142851 A1 as well as the disclosed exemplary embodiments.

A pharmaceutical formulation of the disclosure may comprise, consist essentially of, or consist of (a) a crystalline N-methyl tryptamine derivative according to the disclosure and (b) at least one second active compound selected from a serotonergic drug, a purified psilocybin derivative, a purified cannabinoid, a purified terpene, an adrenergic drug, a dopaminergic drug, a monoamine oxidase inhibitor, a purified erinacine, and a purified hericenone, and (c) a pharmaceutically acceptable excipient. In some embodiments, a crystalline N-methyl tryptamine derivative according to the disclosure, and the second active compound(s) are each present in a therapeutically effective amount using purposefully engineered and unnaturally occurring molar ratios. Exemplary molar ratios of a crystalline N-methyl tryptamine derivative according to the disclosure to the second active compound in a composition of the disclosure include but are not limited to from about 0.1:100 to about 100:0.1, from about 1:100 to about 100:1, from about 1:50 to about 50:1, from about 1:25 to about 25:1, from about 1:20 to about 20:1, from about 1:10 to about 10:1, from about 1:5 to about 5:1, from about 1:2 to about 2:1 or may be about 1:1.

A pharmaceutical formulation of the disclosure may comprise a composition containing a crystalline N-methyl tryptamine derivative according to the disclosure and a serotonergic drug, a purified psilocybin derivative, a purified cannabinoid, or a purified terpene, each present in a therapeutically effective amount using purposefully engineered and unnaturally occurring molar ratios. Published US applications US 2018/0221396 A1 and US 2019/0142851 A1 disclose compositions comprising a combination of a purified psilocybin derivative with a second purified psilocybin derivative, with one or two purified cannabinoids or with a purified terpene. According to this disclosure composition containing a crystalline N-methyl tryptamine derivative according to the disclosure may be used in place of a “purified psilocybin derivative” in the compositions described in US 2018/0221396 A1 and US 2019/0142851 A1. Accordingly, the disclosure provides a pharmaceutical formulation comprising as (a) a crystalline N-methyl tryptamine derivative according to the disclosure and at least one second component selected from (a) a purified psilocybin derivative, (b) a purified cannabinoid, and (c) a purified terpene; and at least one pharmaceutically-acceptable excipient or at least one other adjuvant, as described herein. Such a composition may be a pharmaceutical composition wherein the components are present individually in therapeutic effective amounts or by combination in a therapeutically effective amount to treat a disease, disorder, or condition as described herein.

A serotonergic drug refers to a compound that binds to, blocks, or otherwise influences (e.g., via an allosteric reaction) activity at a serotonin receptor as described in paragraphs [0245]-[0253] of US 2018/0221396 A1 and [0305]-[0311] US 2019/0142851 A1 as well as the disclosed exemplary embodiments. Some exemplary serotonergic drugs include SSRIs and SNRIs. Some examples of specific serotonergic drugs include the following molecules, including any salts, solvates, or polymorphs thereof: 6-allyl-N,N-diethyl-NL; N,N-dibutyl-T; N,N-diethyl-T; N,N-diisopropyl-T; 5-methyoxy-alpha-methyl-T; N,N-dimethyl-T; 2, alpha-dimethyl-T; alpha, N-dimethyl-T; N,N-dipropyl-T; N-ethyl-N-isopropyl-T; alpha-ethyl-T; 6-N,N-Triethyl-NL; 3,4-dihydro-7-methoxy-1-methyl-C; 7-methyoxy-1-methyl-C; N,N-dibutyl-4-hydroxy-T; N,N-diethyl-4-hydroxy-T; N,N-diisopropyl-4-hydroxy-T; N,N-dimethyl-4-hydroxy-T; N,N-dimethyl-5-hydroxy-T; N,N-dipropyl-4-hydroxy-T; N-ethyl-4-hydroxy-N-methyl-T; 4-hydroxy-N-isopropyl-N-methyl-T; 4-hydroxy-N-methyl-N-propyl-T; 4-hydroxy-N,N-tetramethylene-T; ibogaine; N,N-diethyl-L; N-butyl-N-methyl-T; N,N-diisopropyl-4,5-methylenedioxy-T; N,N-diisopropyl-5,6-methylenedioxy-T; N,N-dimethyl-4,5-methylenedioxy-T; N,N-dimethyl-5,6-methylenedioxy-T; N-isopropyl-N-methyl-5,6-methylenedioxy-T; N,N-diethyl-2-methyl-T; 2-N,N-trimethyl-T; N-acetyl-5-methoxy-T; N,N-diethyl-5-methoxy-T; N,N-diisopropyl-5-methoxy-T; 5-methoxy-N,N-dimethyl-T; N-isopropyl-4-methoxy-N-methyl-T; N-isopropyl-5-methoxy-N-methyl-T; 5,6-dimethoxy-N-isopropyl-N-methyl-T; 5-methoxy-N-methyl-T; 5-methoxy-N,N-tetramethylene-T; 6-methoxy-1-methyl-1,2,3,4-tetrahydro-C; 5-methoxy-2-N,N-trimethyl-T; N,N-dimethyl-5-methylthio-T; N-isopropyl-N-methyl-T; alpha-methyl-T; N-ethyl-T; N-methyl-T; 6-propyl-N-L; N,N-tetramethylene-T; tryptamine; 7-methoxy-1-methyl-1,2,3,4-tetrahydro-C; and alpha, N-dimethyl-5-methoxy-T. For additional information regarding these compounds see Shulgin, A. T., & Shulgin, A. (2016). Tihkal: The Continuation. Berkeley, Calif.: Transform Press. In one embodiment, a serotonergic drug is chosen from alprazolam, amphetamine, aripiprazole, azapirone, a barbiturate, bromazepam, bupropion, buspirone, a cannabinoid, chlordiazepoxide, citalopram, clonazepam, clorazepate, dextromethorphan, diazepam, duloxetine, escitalopram, fluoxetine, flurazepam, fluvoxamine, lorazepam, lysergic acid diethylamide, lysergamide, 3,4-methylenedioxymethamphetamine, milnacipran, mirtazapine, naratriptan, paroxetine, pethidine, phenethylamine, psicaine, oxazepam, reboxetine, serenic, serotonin, sertraline, temazepam, tramadol, triazolam, a tryptamine, venlafaxine, vortioxetine, and/or derivatives thereof. In an exemplary embodiment, the serotonergic drug is 3,4-methylenedioxymethamphetamine.

Exemplary psilocybin derivatives include but are not limited to psilocybin itself and the psilocybin derivatives described in paragraphs [0081]-[0109] of US 2018/0221396 A1 and [0082]-[0110] US 2019/0142851 A1 as well as the disclosed exemplary embodiments, incorporated here by reference. In one embodiment, the compositions disclosed herein comprise one or more purified psilocybin derivatives chosen from: [3-(2-dimethylaminoethyl)-1H-indol-4-yl] dihydrogen phosphate; 4-hydroxytryptamine; 4-hydroxy-N,N-dimethyltryptamine; [3-(2-methylaminoethyl)-1H-indol-4-yl]dihydrogen phosphate; 4-hydroxy-N-methyltryptamine; [3-(aminoethyl)-1H-indol-4-yl] dihydrogen phosphate; [3-(2-trimethylaminoethyl)-1H-indol-4-yl] dihydrogen phosphate; and 4-hydroxy-N,N,N-trimethyltryptamine.

Exemplary cannabinoids include but are not limited to the cannabinoids described in paragraphs [0111]-[0145] of US 2018/0221396 A1 and [0112]-[0146] US 2019/0142851 A1 as well as the disclosed exemplary embodiments. Examples of cannabinoids within the context of this disclosure include the following molecules: cannabichromene (CBC); cannabichromenic acid (CBCA); cannabichromevarin (CBCV); cannabichromevarinic acid (CBCVA); cannabicyclol (CBL); cannabicyclolic acid (CBLA); cannabicyclovarin (CBLV); cannabidiol (CBD); cannabidiol monomethylether (CBDM); cannabidiolic acid (CBDA); cannabidiorcol (CBD-C1); cannabidivarin (CBDV); cannabidivarinic acid (CBDVA); cannabielsoic acid B (CBEA-B); cannabielsoin (CBE); cannabielsoin acid A (CBEA-A); cannabigerol (CBG); cannabigerol monomethylether (CBGM); cannabigerolic acid (CBGA); cannabigerolic acid monomethylether (CBGAM); cannabigerovarin (CBGV); cannabigerovarinic acid (CBGVA); cannabinodiol (CBND); cannabinodivarin (CBVD); cannabinol (CBN); cannabinol methylether (CBNM); cannabinol-C2 (CBN-C2); cannabinol-C4 (CBN-C4); cannabinolic acid (CBNA); cannabiorcol (CBN-C1); cannabivarin (CBV); cannabitriol (CBT); cannabitriolvarin (CBTV); 10-ethoxy-9-hydroxy-delta-6a-tetrahydrocannabinol; cannbicitran (CBTC); cannabiripsol (CBR); 8,9-dihydroxy-delta-6a-tetrahydrocannabinol; delta-8-tetrahydrocannabinol (A8-THC); delta-8-tetrahydrocannabinolic acid (A8-THCA); delta-9-tetrahydrocannabinol (THC); delta-9-tetrahydrocannabinol-C4 (THC-C4); delta-9-tetrahydrocannabinolic acid A (THCA-A); delta-9-tetrahydrocannabinolic acid B (THCA-B); delta-9-tetrahydrocannabinolic acid-C4 (THCA-C4); delta-9-tetrahydrocannabiorcol (THC-C1); delta-9-tetrahydrocannabiorcolic acid (THCA-C1); delta-9-tetrahydrocannabivarin (THCV); delta-9-tetrahydrocannabivarinic acid (THCVA); 10-oxo-delta-6a-tetrahydrocannabinol (OTHC); cannabichromanon (CBCF); cannabifuran (CBF); cannabiglendol; delta-9-cis-tetrahydrocannabinol (cis-THC); trihydroxy-delta-9-tetrahydrocannabinol (triOH-THC); dehydrocannabifuran (DCBF); and 3,4,5,6-tetrahydro-7-hydroxy-alpha-alpha-2-trimethyl-9-n-propyl-2,6-metha-no-2H-1-benzoxocin-5-methanol. In one embodiment, the purified cannabinoid is chosen from THC, THCA, THCV, THCVA, CBC, CBCA, CBCV, CBCVA, CBD, CBDA, CBDV, CBVD, CBDVA, CBG, CBGA, CBGV, or CBGVA.

Exemplary terpenes include but are not limited to the terpenes described in paragraphs [0160]-[0238] of US 2018/0221396 A1 and [0161]-[0300] US 2019/0142851 A1 as well as the disclosed exemplary embodiments. In one embodiment, a purified terpene is chosen from acetanisole, acetyl cedrene, anethole, anisole, benzaldehyde, bornyl acetate, borneol, cadinene, cafestol, caffeic acid, camphene, camphor, capsaicin, carene, carotene, carvacrol, carvone, caryophyllene, caryophyllene, caryophyllene oxide, cedrene, cedrene epoxide, cecanal, cedrol, cembrene, cinnamaldehyde, cinnamic acid, citronellal, citronellol, cymene, eicosane, elemene, estragole, ethyl acetate, ethyl cinnamate, ethyl maltol, eucalyptol/1,8-cineole, eudesmol, eugenol, euphol, farnesene, farnesol, fenchone, geraniol, geranyl acetate, guaia-1(10),11-diene, guaiacol, guaiol, guaiene, gurjunene, herniarin, hexanaldehyde, hexanoic acid, humulene, ionone, ipsdienol, isoamyl acetate, isoamyl alcohol, isoamyl formate, isoborneol, isomyrcenol, isoprene, isopulegol, isovaleric acid, lavandulol, limonene, gamma-linolenic acid, linalool, longifolene, lycopene, menthol, methyl butyrate, 3-mercapto-2-methylpentanal, beta-mercaptoethanol, mercaptoacetic acid, methyl salicylate, methylbutenol, methyl-2-methylvalerate, methyl thiobutyrate, myrcene, gamma-muurolene, nepetalactone, nerol, nerolidol, neryl acetate, nonanaldehyde, nonanoic acid, ocimene, octanal, octanoic acid, pentyl butyrate, phellandrene, phenylacetaldehyde, phenylacetic acid, phenylethanethiol, phytol, pinene, propanethiol, pristimerin, pulegone, retinol, rutin, sabinene, squalene, taxadiene, terpineol, terpine-4-ol, terpinolene, thujone, thymol, umbelliferone, undecanal, verdoxan, or vanillin. In one embodiment, a purified terpene is chosen from bornyl acetate, alpha-bisabolol, borneol, camphene, camphor, carene, caryophyllene, cedrene, cymene, elemene, eucalyptol, eudesmol, farnesene, fenchol, geraniol, guaiacol, humulene, isoborneol, limonene, linalool, menthol, myrcene, nerolidol, ocimene, phellandrene, phytol, pinene, pulegone, sabinene, terpineol, terpinolene, or valencene.

As used herein, the term “adrenergic drug” refers to a compound that binds, blocks, or otherwise influences (e.g., via an allosteric reaction) activity at an adrenergic receptor. In one embodiment, an adrenergic drug binds to an adrenergic receptor. In one embodiment, an adrenergic drug indirectly affects an adrenergic receptor, e.g., via interactions affecting the reactivity of other molecules at the adrenergic receptor. In one embodiment, an adrenergic drug is an agonist, e.g., a compound activating an adrenergic receptor. In one embodiment, an adrenergic drug is an antagonist, e.g., a compound binding but not activating an adrenergic receptor, e.g., blocking a receptor. In one embodiment, an adrenergic drug is an effector molecule, e.g., a compound binding to an enzyme for allosteric regulation. In one embodiment, an adrenergic drug acts (either directly or indirectly) at more than one type of receptor (e.g., 5HT, dopamine, adrenergic, acetylcholine, etc.).

In one embodiment, an adrenergic drug is an antidepressant. In one embodiment, an adrenergic drug is a norepinephrine transporter inhibitor. In one embodiment, an adrenergic drug is a vesicular monoamine transporter inhibitor. In one embodiment, an adrenergic drug is chosen from adrenaline, agmatine, amoxapine, aptazapine, atomoxetine, bupropion, clonidine, doxepin, duloxetine, esmirtazpine, mianserin, ketanserin, mirabegron, mirtazapine, norepinephrine, phentolamine, phenylephrine, piperoxan, reserpine, ritodrine, setiptiline, tesofensine, timolol, trazodone, trimipramine, or xylazine.

As used herein, the term “dopaminergic drug” refers to a compound that binds, blocks, or otherwise influences (e.g., via an allosteric reaction) activity at a dopamine receptor. In one embodiment, a dopaminergic drug binds to a dopamine receptor. In one embodiment, a dopaminergic drug indirectly affects a dopamine receptor, e.g., via interactions affecting the reactivity of other molecules at the dopamine receptor. In one embodiment, a dopaminergic drug is an agonist, e.g., a compound activating a dopamine receptor. In one embodiment, a dopaminergic drug is an antagonist, e.g., a compound binding but not activating a dopamine receptor, e.g., blocking a receptor. In one embodiment, a dopaminergic drug is an effector molecule, e.g., a compound binding to an enzyme for allosteric regulation. In one embodiment, a dopaminergic drug acts (either directly or indirectly) at more than one type of receptor (e.g., 5HT, dopamine, adrenergic, acetylcholine, etc.).

In one embodiment, a dopaminergic drug is a dopamine transporter inhibitor. In one embodiment, a dopaminergic drug is a vesicular monoamine transporter inhibitor. In one embodiment, a dopaminergic drug is chosen from amineptine, apomorphine, benzylpiperazine, bromocriptine, cabergoline, chlorpromazine, clozapine, dihydrexidine, domperidone, dopamine, fluphenazine, haloperidol, ketamine, loxapine, methamphetamine, olanzapine, pemoline, perphenazine, pergolide, phencyclidine, phenethylamine, phenmetrazine, pimozide, piribedil, a psychostimulant, reserpine, risperidone, ropinirole, tetrabenazine, or thioridazine.

As used herein, the term “monoamine oxidase inhibitor” (MAOI) refers to a compound that blocks the actions of monoamine oxidase enzymes. In one embodiment, a MAOI inhibits the activity of one or both monoamine oxidase A and monoamine oxidase B. In one embodiment a MAOI is a reversible inhibitor of monoamine oxidase A. In one embodiment a MAOI is a drug chosen from isocarboxazid, phenelzine, or tranylcypromine. In one embodiment, a MAOI is β-carboline, pinoline, harmane, harmine, harmaline, harmalol, tetrahydroharmine, 9-methyl-p-carboline, or 3-carboxy-tetrahydrononharman.

In one embodiment, the compositions and methods disclosed herein include one or more purified erinacine molecules. In one embodiment, the compositions and methods disclosed herein comprise purified erinacine A. In one embodiment, the compositions and methods disclosed herein comprise erinacine B. In one embodiment, the compositions and methods disclosed herein comprise erinacine C. In one embodiment, the compositions and methods disclosed herein comprise erinacine D. In one embodiment, the compositions and methods disclosed herein comprise erinacine E. In one embodiment, the compositions and methods disclosed herein comprise erinacine F. In one embodiment, the compositions and methods disclosed herein comprise erinacine G. In one embodiment, the compositions and methods disclosed herein comprise erinacine H. In one embodiment, the compositions and methods disclosed herein comprise erinacine I. In one embodiment, the compositions and methods disclosed herein comprise erinacine J. In one embodiment, the compositions and methods disclosed herein comprise erinacine K In one embodiment, the compositions and methods disclosed herein comprise erinacine P. In one embodiment, the compositions and methods disclosed herein comprise erinacine Q. In one embodiment, the compositions and methods disclosed herein comprise erinacine R. In one embodiment, the compositions and methods disclosed herein comprise erinacine S.

In one embodiment, the compositions and methods disclosed herein include one or more purified hericenone molecules. In one embodiment, the compositions and methods disclosed herein comprise purified hericenone A. In one embodiment, the compositions and methods disclosed herein comprise purified hericenone B. In one embodiment, the compositions and methods disclosed herein comprise purified hericenone C. In one embodiment, the compositions and methods disclosed herein comprise purified hericenone D. In one embodiment, the compositions and methods disclosed herein comprise purified hericenone E. In one embodiment, the compositions and methods disclosed herein comprise purified hericenone F. In one embodiment, the compositions and methods disclosed herein comprise purified hericenone G. In one embodiment, the compositions and methods disclosed herein comprise purified hericenone H.

Exemplary compositions of a crystalline N-methyl tryptamine derivative according to the disclosure and a second compound selected from a serotonergic drug, a purified psilocybin derivative, a purified cannabinoid, a purified terpene, an adrenergic drug, a dopaminergic drug, a monoamine oxidase inhibitor, a purified erinacine, and a purified hericenone in exemplary molar ratios are shown in Table 1. A crystalline N-methyl tryptamine derivative according to the disclosure may be any one of the exemplary embodiments described above including the crystalline forms as disclosed herein.

TABLE 1

| | Molar ratio of a | Molar ratio of a | Molar ratio of a |
|---|---|---|---|
| | crystalline N-methyl | crystalline N-methyl | crystalline N-methyl |
| | tryptamine | tryptamine | tryptamine |
| | derivative according | derivative according | derivative according |
| | to the disclosure: | to the disclosure: | to the disclosure: |
| Second Compound | second compound | second compound | second compound |
| 3,4- | About 1:100 to about | About 1:25 to about | About 1:5 to about |
| methylenedioxymethamph | 100:1 | 25:1 | 5:1 |
| etamine | | | |
| Citalopram | About 1:100 to about | About 1:25 to about | About 1:5 to about |
| | 100:1 | 25:1 | 5:1 |
| Escitalopram | About 1:100 to about | About 1:25 to about | About 1:5 to about |
| | 100:1 | 25:1 | 5:1 |
| Fluoxetine | About 1:100 to about | About 1:25 to about | About 1:5 to about |
| | 100:1 | 25:1 | 5:1 |
| Paroxetine | About 1:100 to about | About 1:25 to about | About 1:5 to about |
| | 100:1 | 25:1 | 5:1 |
| Sertraline | About 1:100 to about | About 1:25 to about | About 1:5 to about |
| | 100:1 | 25:1 | 5:1 |
| Duloxetine | About 1:100 to about | About 1:25 to about | About 1:5 to about |
| | 100:1 | 25:1 | 5:1 |
| [3-(2-dimethylaminoethyl)- | About 1:100 to about | About 1:25 to about | About 1:5 to about |
| 1H-indol-4-yl] dihydrogen | 100:1 | 25:1 | 5:1 |
| phosphate | | | |
| 4-hydroxytryptamine | About 1:100 to about | About 1:25 to about | About 1:5 to about |
| | 100:1 | 25:1 | 5:1 |
| 4-hydroxy-N,N- | About 1:100 to about | About 1:25 to about | About 1:5 to about |
| dimethyltryptamine | 100:1 | 25:1 | 5:1 |
| [3-(2-methylaminoethyl)- | About 1:100 to about | About 1:25 to about | About 1:5 to about |
| 1H-indol-4-yl] dihydrogen | 100:1 | 25:1 | 5:1 |
| phosphate | | | |
| 4-hydroxy-N- | About 1:100 to about | About 1:25 to about | About 1:5 to about |
| methyltryptamine | 100:1 | 25:1 | 5:1 |
| [3-(aminoethyl)-1H-indol-4- | About 1:100 to about | About 1:25 to about | About 1:5 to about |
| yl] dihydrogen phosphate | 100:1 | 25:1 | 5:1 |
| [3-(2-trimethylaminoethyl)- | About 1:100 to about | About 1:25 to about | About 1:5 to about |
| 1H-indol-4-yl] dihydrogen | 100:1 | 25:1 | 5:1 |
| phosphate | | | |
| 4-hydroxy-N,N,N- | About 1:100 to about | About 1:25 to about | About 1:5 to about |
| trimethyltryptamine | 100:1 | 25:1 | 5:1 |
| THC | About 1:100 to about | About 1:25 to about | About 1:5 to about |
| | 100:1 | 25:1 | 5:1 |
| CBC | About 1:100 to about | About 1:25 to about | About 1:5 to about |
| | 100:1 | 25:1 | 5:1 |
| CBD | About 1:100 to about | About 1:25 to about | About 1:5 to about |
| | 100:1 | 25:1 | 5:1 |
| CBG | About 1:100 to about | About 1:25 to about | About 1:5 to about |
| | 100:1 | 25:1 | 5:1 |
| Myrcene | About 1:100 to about | About 1:25 to about | About 1:5 to about |
| | 100:1 | 25:1 | 5:1 |
| Pinene | About 1:100 to about | About 1:25 to about | About 1:5 to about |
| | 100:1 | 25:1 | 5:1 |
| Caryophyllene | About 1:100 to about | About 1:25 to about | About 1:5 to about |
| | 100:1 | 25:1 | 5:1 |
| Limonene | About 1:100 to about | About 1:25 to about | About 1:5 to about |
| | 100:1 | 25:1 | 5:1 |
| Humulene | About 1:100 to about | About 1:25 to about | About 1:5 to about |
| | 100:1 | 25:1 | 5:1 |
| Linalool | About 1:100 to about | About 1:25 to about | About 1:5 to about |
| | 100:1 | 25:1 | 5:1 |
| Adrenaline | About 1:100 to about | About 1:25 to about | About 1:5 to about |
| | 100:1 | 25:1 | 5:1 |
| Amineptine | About 1:100 to about | About 1:25 to about | About 1:5 to about |
| | 100:1 | 25:1 | 5:1 |
| Erinacine A | About 1:100 to about | About 1:25 to about | About 1:5 to about |
| | 100:1 | 25:1 | 5:1 |
| Hericenone A | About 1:100 to about | About 1:25 to about | About 1:5 to about |
| | 100:1 | 25:1 | 5:1 |
| Phenelzine | About 1:100 to about | About 1:25 to about | About 1:5 to about |
| | 100:1 | 25:1 | 5:1 |

Exemplary pharmaceutical compositions of a crystalline N-methyl tryptamine derivative according to the disclosure and a second compound selected from a serotonergic drug, a purified psilocybin derivative, a purified cannabinoid, a purified terpene, an adrenergic drug, a dopaminergic drug, a monoamine oxidase inhibitor, a purified erinacine, and a purified hericenone and an excipient with exemplary molar ratios of a crystalline N-methyl tryptamine derivative according to the disclosure to the second compound are shown in Table 2. The crystalline N-methyl tryptamine derivative according to the disclosure may be any one of the exemplary embodiments described above including the crystalline forms as disclosed herein.

TABLE 2

| | Molar ratio of a | Molar ratio of a | Molar ratio of a |
|---|---|---|---|
| | crystalline N-methyl | crystalline N-methyl | crystalline N-methyl |
| | tryptamine | tryptamine | tryptamine |
| | derivative according | derivative according | derivative according |
| | to the disclosure: | to the disclosure: | to the disclosure: |
| Second Compound | second compound | second compound | second compound |
| 3,4- | About 1:100 to about | About 1:25 to about | About 1:5 to about |
| methylenedioxymethamph | 100:1 | 25:1 | 5:1 |
| etamine | | | |
| Citalopram | About 1:100 to about | About 1:25 to about | About 1:5 to about |
| | 100:1 | 25:1 | 5:1 |
| Escitalopram | About 1:100 to about | About 1:25 to about | About 1:5 to about |
| | 100:1 | 25:1 | 5:1 |
| Fluoxetine | About 1:100 to about | About 1:25 to about | About 1:5 to about |
| | 100:1 | 25:1 | 5:1 |
| Paroxetine | About 1:100 to about | About 1:25 to about | About 1:5 to about |
| | 100:1 | 25:1 | 5:1 |
| Sertraline | About 1:100 to about | About 1:25 to about | About 1:5 to about |
| | 100:1 | 25:1 | 5:1 |
| Duloxetine | About 1:100 to about | About 1:25 to about | About 1:5 to about |
| | 100:1 | 25:1 | 5:1 |
| [3-(2-dimethylaminoethyl)- | About 1:100 to about | About 1:25 to about | About 1:5 to about |
| 1H-indol-4-yl] dihydrogen | 100:1 | 25:1 | 5:1 |
| phosphate | | | |
| 4-hydroxytryptamine | About 1:100 to about | About 1:25 to about | About 1:5 to about |
| | 100:1 | 25:1 | 5:1 |
| 4-hydroxy-N,N- | About 1:100 to about | About 1:25 to about | About 1:5 to about |
| dimethyltryptamine | 100:1 | 25:1 | 5:1 |
| [3-(2-methylaminoethyl)- | About 1:100 to about | About 1:25 to about | About 1:5 to about |
| 1H-indol-4-yl] dihydrogen | 100:1 | 25:1 | 5:1 |
| phosphate | | | |
| 4-hydroxy-N- | About 1:100 to about | About 1:25 to about | About 1:5 to about |
| methyltryptamine | 100:1 | 25:1 | 5:1 |
| [3-(aminoethyl)-1H-indol-4- | About 1:100 to about | About 1:25 to about | About 1:5 to about |
| yl] dihydrogen phosphate | 100:1 | 25:1 | 5:1 |
| [3-(2-trimethylaminoethyl)- | About 1:100 to about | About 1:25 to about | About 1:5 to about |
| 1H-indol-4-yl] dihydrogen | 100:1 | 25:1 | 5:1 |
| phosphate | | | |
| 4-hydroxy-N,N,N- | About 1:100 to about | About 1:25 to about | About 1:5 to about |
| trimethyltryptamine | 100:1 | 25:1 | 5:1 |
| THC | About 1:100 to about | About 1:25 to about | About 1:5 to about |
| | 100:1 | 25:1 | 5:1 |
| CBC | About 1:100 to about | About 1:25 to about | About 1:5 to about |
| | 100:1 | 25:1 | 5:1 |
| CBD | About 1:100 to about | About 1:25 to about | About 1:5 to about |
| | 100:1 | 25:1 | 5:1 |
| CBG | About 1:100 to about | About 1:25 to about | About 1:5 to about |
| | 100:1 | 25:1 | 5:1 |
| Myrcene | About 1:100 to about | About 1:25 to about | About 1:5 to about |
| | 100:1 | 25:1 | 5:1 |
| Pinene | About 1:100 to about | About 1:25 to about | About 1:5 to about |
| | 100:1 | 25:1 | 5:1 |
| Caryophyllene | About 1:100 to about | About 1:25 to about | About 1:5 to about |
| | 100:1 | 25:1 | 5:1 |
| Limonene | About 1:100 to about | About 1:25 to about | About 1:5 to about |
| | 100:1 | 25:1 | 5:1 |
| Humulene | About 1:100 to about | About 1:25 to about | About 1:5 to about |
| | 100:1 | 25:1 | 5:1 |
| Linalool | About 1:100 to about | About 1:25 to about | About 1:5 to about |
| | 100:1 | 25:1 | 5:1 |
| Adrenaline | About 1:100 to about | About 1:25 to about | About 1:5 to about |
| | 100:1 | 25:1 | 5:1 |
| Amineptine | About 1:100 to about | About 1:25 to about | About 1:5 to about |
| | 100:1 | 25:1 | 5:1 |
| Erinacine A | About 1:100 to about | About 1:25 to about | About 1:5 to about |
| | 100:1 | 25:1 | 5:1 |
| Hericenone A | About 1:100 to about | About 1:25 to about | About 1:5 to about |
| | 100:1 | 25:1 | 5:1 |
| Phenelzine | About 1:100 to about | About 1:25 to about | About 1:5 to about |
| | 100:1 | 25:1 | 5:1 |

An “effective amount” or a “therapeutically effective amount” of a crystalline N-methyl tryptamine derivative according to the disclosure is generally in the range of about 0.1 to about 100 mg daily (oral dose), of about 0.1 to about 50 mg daily (oral dose) of about 0.25 to about 25 mg daily (oral dose), of about 0.1 to about 5 mg daily (oral dose) or of about 0.5 to about 2.5 mg daily (oral dose). The actual amount required for treatment of any particular patient may depend upon a variety of factors including, for example, the disease being treated and its severity; the specific pharmaceutical composition employed; the age, body weight, general health, sex, and diet of the patient; the mode of administration; the time of administration; the route of administration; and the rate of excretion; the duration of the treatment; any drugs used in combination or coincidental with the specific compound employed; and other such factors well known in the medical arts. These factors are discussed in Goodman and Gilman's “The Pharmacological Basis of Therapeutics,” Tenth Edition, A. Gilman, J. Hardman and L. Limbird, eds., McGraw-Hill Press, 155-173 (2001), which is incorporated herein by reference. A crystalline N-methyl tryptamine derivative according to the disclosure, compositions and pharmaceutical compositions containing it may be used in combination with other agents that are generally administered to a patient being treated for psychological and other disorders discussed above. They may also be co-formulated with one or more of such agents in a single pharmaceutical composition.

Depending on the type of composition or pharmaceutical composition, the excipient or pharmaceutically acceptable carrier may be chosen from any one or a combination of carriers known in the art. The choice of the pharmaceutically acceptable carrier depends upon the pharmaceutical form and the desired method of administration to be used. Preferred carriers include those that do not substantially alter the crystalline N-methyl tryptamine derivative or produce undesirable biological effects or otherwise interact in a deleterious manner with any other component(s) of the pharmaceutical composition.

The compositions or pharmaceutical compositions of the disclosure may be prepared by methods know in the pharmaceutical formulation art, for example, see Remington's Pharmaceutical Sciences, 18th Ed., (Mack Publishing Company, Easton, Pa., 1990), which is incorporated herein by reference. In a solid dosage form, the crystalline N-methyl tryptamine derivative may be admixed with at least one pharmaceutically acceptable excipient such as, for example, sodium citrate or dicalcium phosphate or (a) fillers or extenders, such as, for example, starches, lactose, sucrose, glucose, mannitol, and silicic acid, (b) binders, such as, for example, cellulose derivatives, starch, alginates, gelatin, polyvinylpyrrolidone, sucrose, and gum acacia, (c) humectants, such as, for example, glycerol, (d) disintegrating agents, such as, for example, agar-agar, calcium carbonate, potato or tapioca starch, alginic acid, croscarmellose sodium, complex silicates, and sodium carbonate, (e) solution retarders, such as, for example, paraffin, (f) absorption accelerators, such as, for example, quaternary ammonium compounds, (g) wetting agents, such as, for example, cetyl alcohol, and glycerol monostearate, magnesium stearate and the like (h) adsorbents, such as, for example, kaolin and bentonite, and (i) lubricants, such as, for example, talc, calcium stearate, magnesium stearate, solid polyethylene glycols, sodium lauryl sulfate, or mixtures thereof. In the case of capsules, tablets, and pills, the dosage forms may also comprise buffering agents.

Excipients or pharmaceutically acceptable adjuvants known in the formulation art may also be used in the pharmaceutical compositions of the disclosure. These include, but are not limited to, preserving, wetting, suspending, sweetening, flavoring, perfuming, emulsifying, and dispensing agents. Prevention of the action of microorganisms may be ensured by inclusion of various antibacterial and antifungal agents, for example, parabens, chlorobutanol, phenol, sorbic acid, and the like. It may also be desirable to include isotonic agents, for example, sugars, sodium chloride, and the like. If desired, a composition or a pharmaceutical composition of the disclosure may also contain minor amounts of auxiliary substances such as wetting or emulsifying agents, pH buffering agents, antioxidants, and the like, such as, for example, citric acid, sorbitan monolaurate, triethanolamine oleate, butylated hydroxytoluene, etc.

Solid dosage forms as described above may be prepared with coatings and shells, such as enteric coatings and others well known in the art. They may contain pacifying agents and can also be of such composition that they release the active compound or compounds in a certain part of the intestinal tract in a delayed manner. Non-limiting examples of embedded compositions that may be used are polymeric substances and waxes. The active compounds may also be in microencapsulated form, if appropriate, with one or more of the above-mentioned excipients.

Suspensions, in addition to the active compounds, may contain suspending agents, such as, for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Solid dosage forms for oral administration, which includes capsules, tablets, pills, powders, and granules, may be used. In such solid dosage forms, the active compound may be mixed with at least one inert, pharmaceutically acceptable excipient (also known as a pharmaceutically acceptable carrier).

Administration of a crystalline N-methyl tryptamine derivative according to the disclosure in pure form, with a permeation enhancer, with stabilizers (e.g. antioxidants), or in an appropriate pharmaceutical composition may be carried out via any of the accepted modes of administration or agents for serving similar utilities. Thus, administration may be, for example, orally, buccally, nasally, parenterally (intravenous, intramuscular, or subcutaneous), topically, transdermally, intravaginally, intravesically, or intrasystemically, in the form of solid, semi-solid, lyophilized powder, liquid dosage forms, such as, for example, tablets, suppositories, pills, soft elastic and hard gelatin capsules, powders, suspensions, or aerosols, or the like, such as, for example, in unit dosage forms suitable for simple administration of precise dosages. One route of administration may be oral administration, using a convenient daily dosage regimen that can be adjusted according to the degree of severity of the disease-state to be treated.

### EXAMPLES

Single Crystal X-Ray Diffraction (SCXRD) Characterization:

Data were collected on a Bruker D8 Venture CMOS Diffractometer equipped with an Oxford Cryosystems Cryostream cooling device and using Mo Kα radiation. Structures were solved using the Bruker SHELXTL program and refined with the SHELXTL program as part of the Bruker SHELXTL suite, or OLEX2 software. Unless otherwise stated, hydrogen atoms attached to carbon were placed geometrically and allowed to refine with a riding isotropic displacement parameter. Hydrogen atoms attached to a heteroatom were located in a difference Fourier synthesis and were allowed to refine freely with an isotropic displacement parameter.

### Example 1: Preparation and Characterization of Crystalline N-Methyl-N-Propyltryptamine (MPT)

Crystal Preparation

Single crystals suitable for X-ray analysis were obtained from the slow evaporation of a methylene chloride solution of a commercial sample of N-methyl-N-propyltryptamine (MPT) (The Indole Shop, Canada).

Single Crystal X-Ray Diffraction (SCXRD) Characterization

The single crystal data and structure refinement parameters for the crystalline structure measured at 200 K are reported in Table 3, below.

TABLE 3
Experimental Details

| Crystal Data | |
|---|---|
| Chemical formula | C₁₄H₂₀N₂ |
| Mᵣ | 216.32 |
| Crystal system, space group | Orthorhombic, Pbca |
| Temperature(K) | 200 |
| a, b, c (Å) | 13.5715 (11), 12.4352 (10), 15.1627(12) |
| V(Å³) | 2558.9 (4) |
| Z | 8 |
| Radiation Type | Mo Ka |
| μ(mm⁻¹) | 0.07 |
| Crystalsize(mm) | 0.28 × 0.20 × 0.13 |
| Data collection | |
| Diffractometer | Bruker D8 Venture CMOS |
| Absorption correction | Multi-scan (SADABS; Bruker,2016) |
| Tₘᵢₙ, Tₘₐₓ | 0.713, 0.745 |
| No. of measured, | |
| independent and observed | |
| [I > 2σ(I)] reflections | 47431, 2350, 1942 |
| Rᵢₙₜ | 0.048 |
| (sinθ/λ)ₘₐₓ(Å⁻¹) | 0.604 |
| Refinement | |
| R[F² > 2σ(F²)], wR(F²), S | 0.036, 0.095, 1.05 |
| No. of reflections | 2350 |
| No. of parameters | 152 |
| H-atom treatment | H atoms treated by a mixture of |
| | independent and constrained refinement |
| Δρ_{max,} Δρₘᵢₙ(eÅ⁻³) | 0.17,-0.16 |

Computer programs: APEX3 and SAINT (Bruker, 2016), SHELXT2014 (Sheldrick, 2015a), SHELXL2014 (Sheldrick, 2015b), pubICIF (Westrip, 2010) and OLEX2 (Dolamanov et al, 2009).

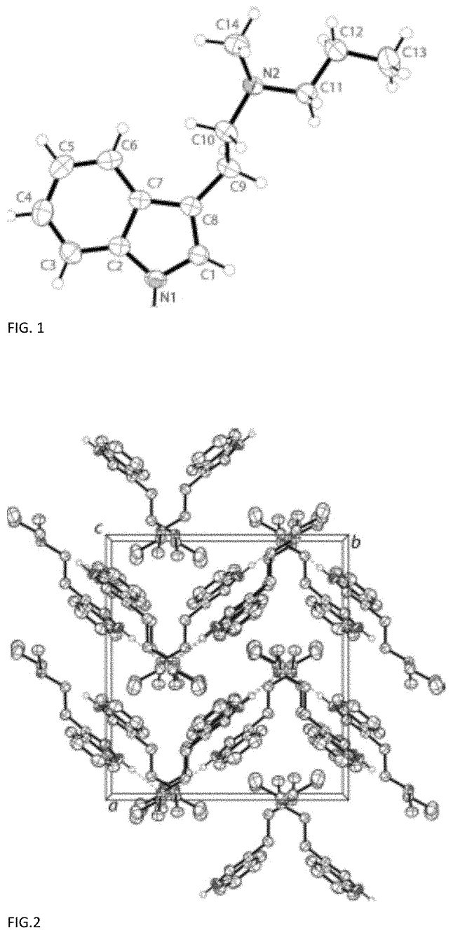

The molecular structure of crystalline MPT according to the disclosure is shown in FIG. 1 with atomic labelling. Displacement ellipsoids are drawn at the 50% probability level. FIG. 2 shows the crystal packing of crystalline MPT, viewed along the c axis. The N—H—N hydrogen bonds are shown as dashed lines. Displacement ellipsoids are drawn at the 50% probability level. H atoms not involved in hydrogen bonding have been omitted for clarity.

In the solid-state structure of MPT, there is a single molecule in the symmetric unit, with an indole group that demonstrates a mean deviation from planarity of 0.015 Å (FIG. 1). The metrical parameters are consistent with the previously reported structure of DMT (Falkenberg, 1972) and other dialkyltryptamines (Chadeayne et al., 2019a,b; Petcher & Weber, 1974; Weber & Petcher, 1974). The tryptamine molecules are held together in an infinite one-dimensional chain along [010] through N—HN hydrogen bonds connecting the indole N atom to the mine N atom (Table 1, FIG. 2). In the structure of N,N-dimethyltryptamine (DMT), there are similar hydrogen bonds, but they hold molecules together as dimers rather than in a chain. There are no π-π interactions observed in the crystalline DMT structure.

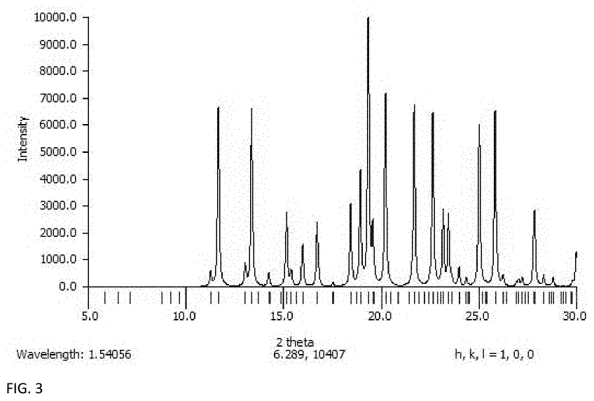

FIG. 3 is a simulated X-ray powder diffraction pattern (XRPD) for crystalline MPT generated from its single crystal data. Characteristic peaks identifying crystalline MPT include peaks at 11.7, 13.4 and 19.4° 2θ±0.2° 2θ.

### Example 2: Preparation and Characterization of Crystalline N-Methyl-N-Isopropyltryptammonium Fumarate (MiPT Fumarate) and 4-Hydroxy-N-Methyl-N-Isopropyltryptammonium Fumarate Monohydrate (HO-MiPT Fumarate Monohydrate)

Crystal Preparation

Single crystals suitable for X-ray analysis were obtained from the slow evaporation of aqueous solutions of commercial samples of N-methyl-N-isopropyltryptammonium fumarate and 4-hydroxy-N-methyl-N-isopropyltryptammonium fumarate monohydrate (The Indole Shop, Canada).

Single Crystal X-Ray Diffraction (SCXRD) Characterization

The single crystal data and structure refinement parameters for the crystalline structure measured at 200 K are reported in Table 4, below.

TABLE 4
Experimental Details

| | MiPT | 4-HO-MiPT |
|---|---|---|
| Crystal Data | | |
| Chemical formula | C₄H₃O₄•C₁₄H₂₁N₂ | C₁₄H₂₁N₂O•H₂O•C₄H₃O₄ |
| Mᵣ | 332.39 | 366.41 |
| Crystal system, space group | Monoclinic, Plje | Monoclinic, C2/c |
| Temperature(K) | 200 | 200 |
| a, b,c(Å) | 9.852(2), 12.789(2), 14.875(3) | 29.507 (3), 8.7445 (8), 17.3659 (18) |
| β (°) | 106.932 (7) | 123.389 (3) |
| V(Å³) | 1793.0 (6) | 3741.2 (7) |
| Z | 4 | 8 |
| Radiation Type | Mo Kα | Mo Kα |
| μ(mm⁻¹) | 0.09 | 0.10 |
| Crystal size (mm) | 0.20 × 0.18 × 0.05 | 0.30 × 0.25 × 0.20 |
| Data collection | | |
| Diffractometer | BrukerD8VentureCMOS | Bruker D8 VentureCMOS |
| Absorption correction | Multi-scan | Multi-scan |
| | (SADABS; Bruker,2016) | (SADABS; Bruker,2016) |
| Tₘᵢₙ, Tₘₐₓ | 0.687, 0.745 | 0.719, 0.745 |
| No. of measured, | | |
| independent and observed | | |
| [I > 2σ(I)] reflections | 36899, 3297, 2605 | 70395, 3458, 2978 |
| Rᵢₙₜ | 0.052 | 0.041 |
| (sinθ/λ)ₘₐₓ(Å⁻¹) | 0.604 | 0.604 |
| Refinement | | |
| R[F² > 2σ(F²)], wR(F²), S | 0.049, 0.127, 1.06 | 0.041, 0.096, 1.08 |
| No. of reflections | 3297 | 3458 |
| No. of parameters | 240 | 320 |
| No. of restraints | 8 | 12 |
| H-atom treatment | H atoms treated by a mixture of | H atoms treated by a mixture of |
| | independent and constrained | independent and constrained |
| | refinement | refinement |
| Δρₘₐₓ, Δρₘᵢₙ(eÅ⁻³) | 0.26, −0.26 | 0.22, −0.20 |

Computer programs: Bruker APEX3, Bruker SAINT, SHELXS97(Sheldrick2008), XL(Sheldrick, 2008),Olex2 (Dolomanovetal., 2009).

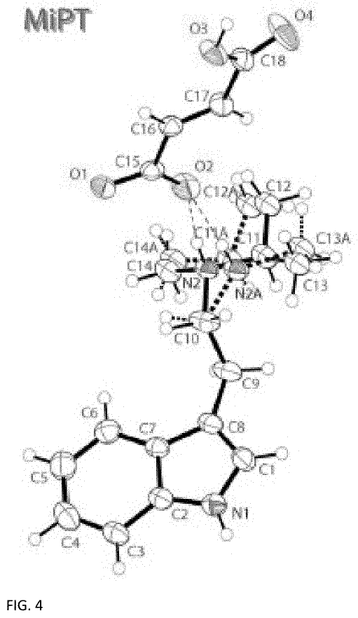

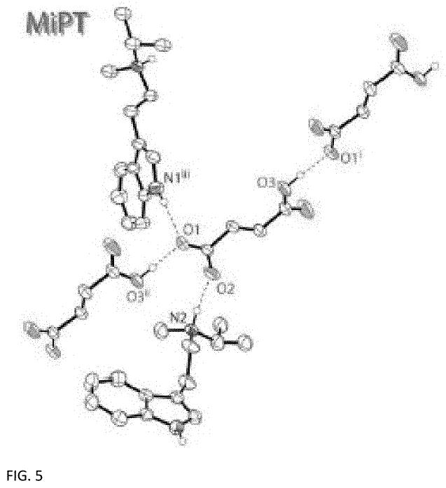

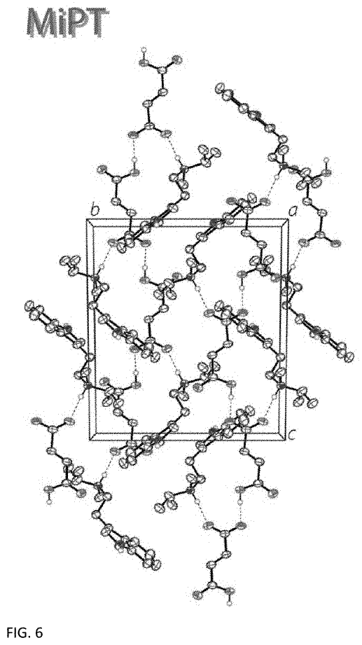

The molecular structure of crystalline MiPT fumarate according to the disclosure is shown in FIG. 4 with atomic labelling, and the hydrogen bonding of the fumarate ion in the structure of crystalline MiPT fumarate is shown in FIG. 5. Displacement ellipsoids are drawn at the 50% probability level. The symmetric unit contains one N-methyl-N-isopropyltryptammonium (C₁₄H₂₁N₂⁺) cation and one 3-carboxyacrylate (C₄H₃O₄⁻) anion. The indole ring system of the cation is near planar with an r.m.s. deviation from planarity of 0.006 Å. The singly protonated fumarate anion is in the trans configuration and is slightly distorted from planarity with an r.m.s. deviation of 0.133 Å and a carboxylate twist angle of 18.370 (5). The N-methyl-N-isopropylammonium group is disordered over two orientations in a 0.630 (3):0.370 (3) ratio.

In the extended crystal structure of MiPT fumarate, the N-methyl-N-isopropylamine and fumarate ions are linked into infinite two-dimensional networks lying parallel to the (010) plane through N—H—O and O—H—O hydrogen bonds. The proton of themmonium cation forms a hydrogen bond with one of the oxygen atoms of the deprotonated —CO₂⁻ group of the 3-carboxyacrylate ion. The carboxylic acid proton forms a hydrogen bond with an oxygen atom of an adjacent 3-carboxyacrylate anion. The N-H grouping of the indole ring also hydrogen bonds to one of the oxygen atoms of the 3-carboxyacrylate anion.

FIG. 6 shows the crystal packing of crystalline MiPT fumarate, viewed along the axis. The hydrogen bonds are shown as dashed lines. Displacement ellipsoids are drawn at the 50% probability level. H atoms not involved in hydrogen bonding have been omitted for clarity.

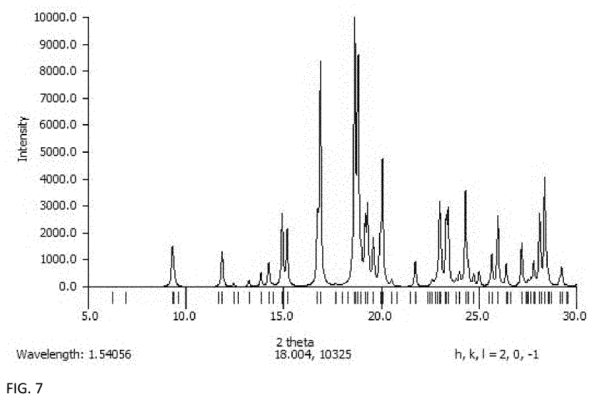

FIG. 7 is a simulated X-ray powder diffraction pattern (XRPD) for crystalline MiPT fumarate generated from its single crystal data. Characteristic peaks identifying crystalline MiPT include peaks at 11.9, 14.9 and 16.9° 2θ±0.2° 2θ.

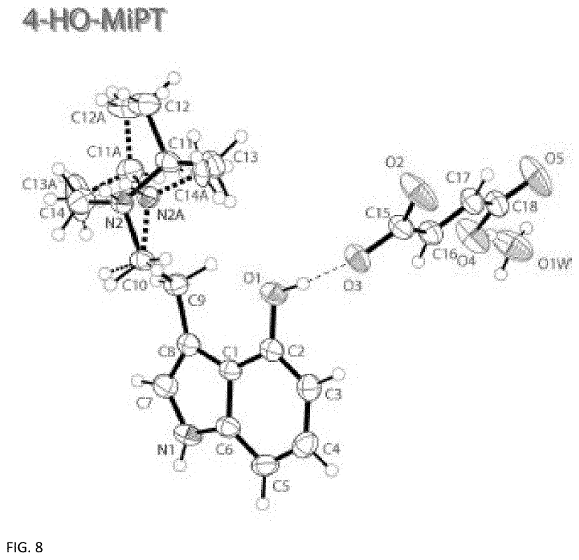

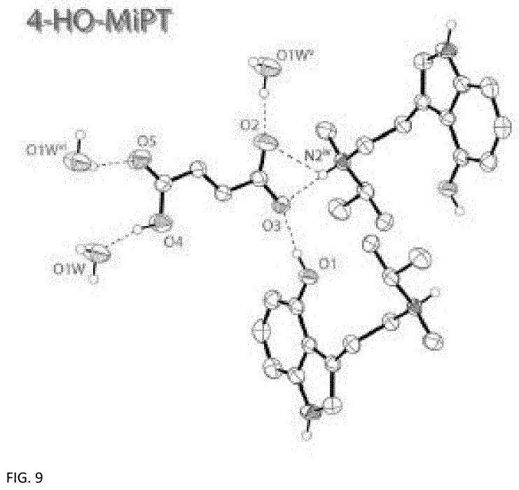

The molecular structure of crystalline HO-MiPT fumarate monohydrate according to the disclosure is shown in FIG. 8 with atomic labelling, and the hydrogen bonding of the fumarate ion in the structure of crystalline MiPT fumarate is shown in FIG. 9.

Displacement ellipsoids are drawn at the 50% probability level. The symmetric unit contains one 4-hydroxy-N-methyl-N-isopropyltryptammonium (C₁₄H₂₁N₂O⁺) cation, one 3-carboxyacrylate anion and one water molecule of crystallization. The indole ring system of the cation is close to planar with an r.m.s. deviation of 0.021 Å. The singly protonated fumarate anion is also near planar with an r.m.s. deviation of 0.049 Å. The N-methyl-N isopropylammonium group shows a similar disorder to the MiPT structure over two orientations in a 0.775 (5):0.225 (5) ratio.

In the structure of 4-HO-MiPT fumarate monohydrate, there are N—H—O and O—H—O hydrogen bonds that link together the cations and anions as well as the water molecules of crystallization. The result is a two-dimensional network lying parallel to the (201) plane. The proton of themmonium cation forms a bifurcated N—H—(O,O) hydrogen bond with the deprotonated —CO₂ group of the 3-carboxyacrylate ion. The hydrogen of the hydroxy group also hydrogen bonds to the same oxygen atom of thenion. The carboxylic acid proton hydrogen bonds with a water molecule in the structure. Two other water molecules form hydrogen bonds with two different oxygen atoms of thenion.

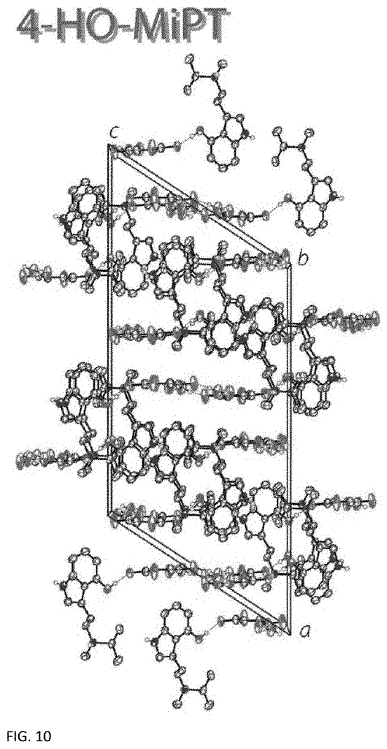

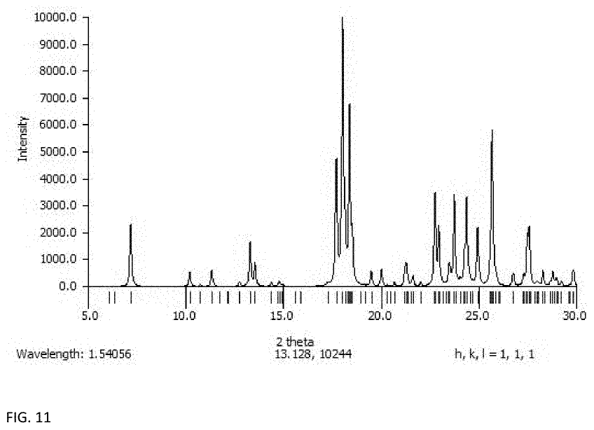

FIG. 10 shows the crystal packing of crystalline HO-MiPT fumarate monohydrate, viewed along the b axis. The hydrogen bonds are shown as dashed lines. Displacement ellipsoids are drawn at the 50% probability level. H atoms not involved in hydrogen bonding have been omitted for clarity.

FIG. 11 is a simulated X-ray powder diffraction pattern (XRPD) for HO-MiPT fumarate monohydrate generated from its single crystal data. Characteristic peaks identifying crystalline HO-MiPT include peaks at 7.2, 13.3 and 18.0° 2θ±0.2° 2θ.

The MiPT structure described above is a derivative of DMT (N,N-dimethyltryptamine), which has been structurally characterized (Falkenberg, 1972), as well as its close derivative MPT, N-methyl-N-propyltryptamine (Chadeayne et al. 2019b). In both cases, these were crystallized as free bases, while MiPT is the fumarate salt. In the case of 4-HO-MiPT, the most closely related molecule is psilocin, which has been structurally characterized (Petcher & Weber, 1974), as well as psilocybin (Weber & Petcher, 1974). Psilocin was reported as the free base and psilocybin was reported as a zwitterionic molecule, while the structure of 4-HO-MiPT reported here is the hydrated fumarate salt. Two different ionic structures of the 4-acetoxy derivative of DMT have been reported as fumarate salts (Chadeayne et al. 2019a,c). The metrical parameters of the tryptammonium cations for MiPT and 4-HOMiPT are consistent with those of the other tryptammonium structures reported.

### Example 3: Preparation and Characterization of Crystalline Form 1 of N,N,N-Trimethyltryptammonium Iodide (TMT Iodide)

Synthesis

162 mg of tryptamine, 322 mg of sodium carbonate and 0.5 mL of iodomethane were dissolved in 10 mL of isopropanol. The solution was heated at reflux for twelve hours under an atmosphere of nitrogen. Solvent was removed in vacuo to yield a yellow powder.

Crystallization

Single crystals suitable for X-ray diffraction were grown from the slow evaporation of an acetone/ethanol solution.

Single Crystal Characterization

The single crystal data and structure refinement parameters for the crystalline form 1 structure of TMT iodide are reported in Table 5, below.

TABLE 5
Crystal data

| Chemical formula | I•C₁₃H₁₉N₂ |
|---|---|
| Mᵣ | 330.20 |
| Crystal system, space group | orthorhombic, P2i2i2i |
| Temperature (K) | 297(2) |
| a, b, c (Å) | 9.4322(6), 11.4021(7), 13.3328(7) |
| α (°) | 90 |
| β (°) | 90 |
| | 90 |
| V (Å3) | 1433.90(15) |
| Z | 4 |
| F(000) | 656 |
| Dₓ (Mg m⁻³) | 1.530 |
| Radiation type | Mo Kα |
| λ (Å) | 0.71073 |
| θ (°) | 2.65-26.34 |
| μ (mm⁻¹) | 2.212 |
| Crystal size (mm) | 0.29 × 0.17 × 0.13 |
| Crystal description | BLOCK |
| Crystal color | colourless |
| Data collection | |
| Diffractometer | Bruker APEX-11 CCD |
| Absorption correction | Multi-scan SADABS (Bruker, 2016) was used. wR2(int) |
| | was 0.0668 before and 0.0472 after correction. The |
| | Ratio of minimum to maximum transmission is 0.8632. |
| | The λ/2 correction factor is not present. |
| Tₘᵢₙ, Tₘₐₓ | 0.6434, 0.7454 |
| No. of measured, independent, and | 43413, 2881, 2637 |
| observed [I > 2σ(I)] reflections | |
| Rᵢₙₜ | 0.0223 |
| θₘₐₓ, θₘᵢₙ (°) | 26.380, 2.645 |
| h, k, I | −11 → 11, −14 → 14, −16 → 16 |
| Refinement | |
| R[F² > 2σ(F²)], wR(F²), S | 0.0165, 0.0380, 1.098 |
| No. of reflections | 2881 |
| No. of parameters | 152 |
| No. of restraints | 0 |
| Absolute structure | Flack x determined using 1097 quotients [(I+) − (I −)]/ |
| | [(I+) + (I−)] (Parsons, Flack and Wagner, Acta Cryst. |
| | B69 (2013) 249-259). |
| Absolute structure parameter | −0.016(5) |
| H-atom treatment | H atoms treated by a mixture of independent and |
| | constrained refinement |
| w | w=1/[σ²(Fₒ²)+(0.0090P)² + 0.6208P] where P=(Fₒ²+2F_{c}²)/3 |
| (Δ/σ)ₘₐₓ | 0.002 |
| Δρₘₐₓ, Δρₘᵢₙ (e Å⁻³) | 0.388, -0.360 |

Data collection: Bruker APEX32; cell refinement: Bruker SAINT; data reduction: Bruker SAINT; program(s) used to solve structure: SHELXS97 (Sheldrick 2008); program(s) used to refine structure: SHELXL 2018/3 (Sheldrick, 2015); molecular graphics: Olex2 1.3 (Dolomanov et al., 2009); software used to prepare material for publication: Olex2 1.3 (Dolomanov et al., 2009).

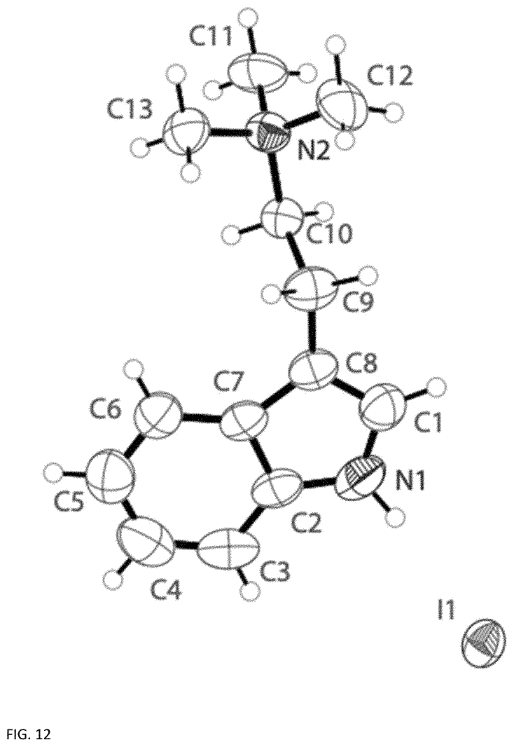

FIG. 12 shows the molecular structure of crystalline form 1 of TMT iodide, showing the atomic labeling.

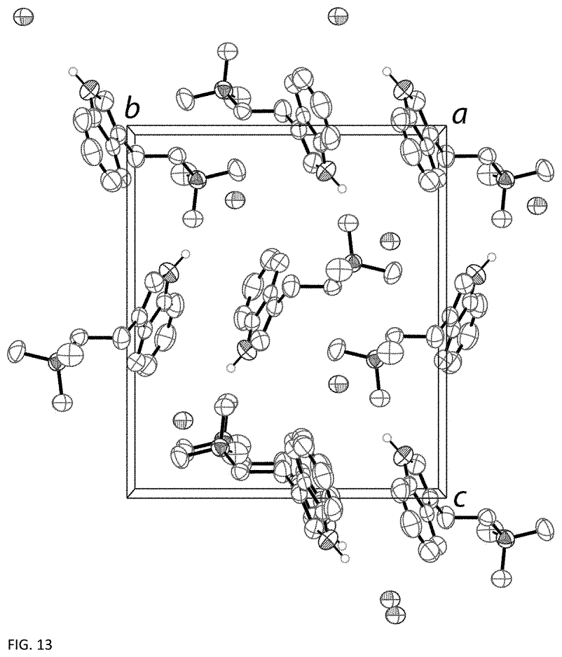

FIG. 13 shows the unit cell of crystalline form 1 of TMT iodide along the a-axis.

Simulated Powder X-Ray Diffraction (PXRD) Pattern

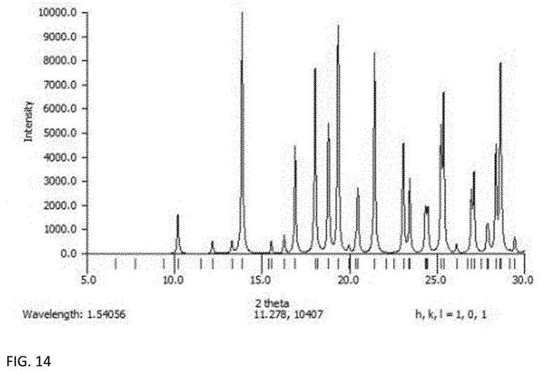

FIG. 14 shows a simulated X-ray powder diffraction pattern (XRPD) for crystalline form 1 of TMT iodide generated from its single crystal data. Table 6 lists the angles, °2θ 0.2° 2θ, and d-spacing of the peaks identified in the experimental XRPD pattern of FIG. 14. The entire list of peaks, or a subset thereof, may be sufficient to characterize the cocrystal. For example, the cocrystal may be characterized by at least two peaks selected from the peaks at 10.19, 13.86, and 16.89° 2θ±0.2° 2θ or their corresponding d-spacing as well as by an XRPD pattern substantially similar to FIG. 14.

TABLE 6

| d-spacing (Å) | 2(Theta deg) | Intensity |
|---|---|---|
| 8.67 | 10.20 | 7494 |
| 7.70 | 11.48 | 80 |
| 7.27 | 12.17 | 3566 |
| 6.67 | 13.27 | 3889 |
| 6.38 | 13.87 | 86954 |
| 5.75 | 15.38 | 2 |
| 5.70 | 15.53 | 5775 |
| 5.44 | 16.27 | 9144 |
| 5.24 | 16.90 | 57016 |
| 4.91 | 18.04 | 112537 |
| 4.88 | 18.17 | 711 |
| 4.72 | 18.80 | 85311 |
| 4.58 | 19.36 | 161017 |
| 4.45 | 19.95 | 5085 |
| 4.36 | 20.36 | 7627 |
| 4.33 | 20.48 | 50628 |
| 4.14 | 21.43 | 48450 |
| 4.14 | 21.44 | 127160 |
| 4.02 | 22.09 | 479 |
| 3.94 | 22.56 | 1021 |
| 3.85 | 23.08 | 111762 |
| 3.79 | 23.44 | 77328 |
| 3.66 | 24.33 | 35906 |
| 3.65 | 24.38 | 24142 |
| 3.63 | 24.48 | 45661 |
| 3.53 | 25.24 | 142226 |
| 3.51 | 25.38 | 142454 |
| 3.51 | 25.39 | 45653 |
| 3.41 | 26.12 | 11603 |
| 3.33 | 26.72 | 319 |
| 3.30 | 26.98 | 80954 |
| 3.29 | 27.12 | 109838 |
| 3.23 | 27.56 | 1 |
| 3.20 | 27.86 | 34284 |
| 3.19 | 27.94 | 29758 |
| 3.14 | 28.38 | 161478 |
| 3.12 | 28.62 | 211645 |
| 3.11 | 28.67 | 142926 |
| 3.06 | 29.16 | 3024 |
| 3.03 | 29.45 | 17571 |
| 3.03 | 29.46 | 10455 |

### REFERENCES

1. Aixala', M., Dos Santos, R. G., Hallak, J. E. C. & Bouso, J. C. (2018). ACS Chem. Neurosci. 9, 2304-2306.
2. Bradley, R. J. & Johnston, V. S. (1970). Origin and Mechanism of Hallucinations, edited by W. Keup, pp. 333-344. New York: Plenum Press.
3. Brandt, S. D., Freeman, S., Fleet, I. A., McGagh, P. & Alder, J. F. (2005). Analyst, 130, 330-344.
4. Bruker (2016). APEX3, SAINT, and SADABS. Bruker AXS Inc., Madison, Wis., USA.
5. Cameron, L. P. & Olson, D. E. (2018). ACS Chem. Neurosci. 9, 2344-2357.
6. Cameron, L. P., Benson, C. J., DeFelice, B. C., Fiehn, O. & Olson, D. E. (2019). ACS Chem. Neurosci. In the press. http://doi.org/10.1021/acschemneuro.8b00692.
7. Carhart-Harris, R. L., Bolstridge, M., Rucker, J., Day, C. M., Erritzoe, D., Kaelen, M., Bloomfield, M., Rickard, J. A., Forbes, B., Feilding, A., Taylor, D., Pilling, S., Curran, V. H. & Nutt, D. J. (2016). Lancet Psychiatr. 3, 619-627.
8. Carhart-Harris, R. L. & Goodwin, G. M. (2017). Neuropsychopharmacology, 42, 2105-2113.
9. Chadeayne, A. R., Golen, J. A. & Manke, D. R. (2019a). Acta Cryst. E75, 900-902.
10. Chadeayne, A. R., Golen, J. A. &Manke, D. R. (2019b). IUCrData, 4, x190962.
11. Chadeayne, A. R., Golen, J. A. & Manke, D. R. (2019c). Psychedelic Science Review, https://psychedelicreview. com/the-crystal-structure-of-4-aco-dmt-fumarate/
12. Dinis-Oliveira, R. J. (2017). Drug Metab. Rev. 49, 84-91.
13. Dolomanov, O. V., Bourhis, L. J., Gildea, R. J., Howard, J. A. K. & Puschmann, H. (2009). J. Appl. Cryst. 42, 339-341.
14. Falkenberg, G. (1972). Acta Cryst. B28, 3075-3083.
15. Fontanilla, D., Johannessen, M., Hajipour, A. R., Cozzi, N. V., Jackson, M. B. & Ruoho, A. E. (2009). Science, 323, 934-937.
16. Gilman, A.; Hardman, J.; Limbird, L. eds., Goodman and Gilman's “The Pharmacological Basis of Therapeutics,” Tenth Edition, McGraw-Hill Press, 155-173 (2001).
17. Johnson, M. W. & Griffiths, R. R. (2017). Neurotherapeutics, 14, 734-740.
18. Manske, R. H. F. (1931). Can. J. Res. 5, 592-600.
19. McKenna, D. J., Repke, D. B., Lo, L. & Peroutka, S. J. (1990). Neuropharmacology, 29, 193-198.
20. Nichols, D. E. (2012). WIREs Membr. Transp. Signal. 1, 559-579.
21. Nichols, D. E. (2016). Pharmacol. Rev. 68, 264-355.
22. Passie, T., Seifert, J., Schneider, U. & Emrich, H. M. (2002). Addict. Biol. 7, 357-364.
23. Petcher, T. J. & Weber, H. P. (1974). J. Chem. Soc. Perkin Trans. II, pp. 946-948.
24. Repke, D. B., Ferguson, W. J. & Bates, D. K. (1981). J. Heterocycl. Chem. 18, 175-179.
25. Repke, D. B., Grotjahn, D. B. & Shulgin, A. T. (1985). J. Med. Chem. 28, 892-896.
26. Sheldrick, G. M. (2008). Acta Cryst. A64, 112-122.
27. Sheldrick, G. M. (2015a). Acta Cryst. A71, 3-8.
28. Sheldrick, G. M. (2015b). Acta Cryst. C71, 3-8.
29. Weber, H. P. & Petcher, T. J. (1974). J. Chem. Soc. Perkin Trans. II, pp. 942-946.
30. Westrip, S. P. (2010). J. Appl. Cryst. 43, 920-925.

1. Crystalline [2-(1H-indol-3-yl)ethyl]trimethylazanium iodide (N,N,N-trimethyltryptammonium iodide).

2. Crystalline form 1 of [2-(1H-indol-3-yl)ethyl]trimethylazanium iodide (N,N,N-trimethyltryptammonium iodide).

3. Crystalline form 1 of N,N,N-trimethyltryptammonium iodide according to claim 2, characterized by at least one of:
an orthorhombic crystal system at a temperature of about 297 K;
a P2₁2₁2₁ space group at a temperature of about 297 K;
unit cell dimensions a=9.4322(6) Å, b=11.4021(7) Å, c=13.3328(7) Å, α=90°, β=90°, and γ=90°;
an X-ray powder diffraction pattern substantially similar to FIG. 14; or
an X-ray powder diffraction pattern characterized by at least two peaks selected from 10.2, 13.9, 16.9, and 21.4° 2θ±0.2° 2θ.

4. A composition comprising crystalline N,N,N-trimethyltryptammonium iodide according to claim 1 and an excipient.

5. A composition comprising crystalline N,N,N-trimethyltryptammonium iodide according to claim 1 as a first component and a second component selected from at least one of (a) a serotonergic drug, (b) a purified psilocybin derivative, (c) a purified cannabinoid, (d) a purified terpene, (e) an adrenergic drug, (f) a dopaminergic drug, (g) a monoamine oxidase inhibitor, (h) a purified erinacine, and (i) a purified hericenone.

6. A method of preventing or treating a psychological disorder comprising the step of:
administering to a subject in need thereof a therapeutically effective amount of crystalline N,N,N-trimethyltryptammonium iodide according to claim 1.

7. A method of preventing or treating a psychological disorder comprising the step of:
administering to a subject in need thereof a composition according to claim 4.

8. A method of preventing or treating inflammation and/or pain comprising the step of:
administering to a subject in need thereof a therapeutically effective amount of crystalline N,N,N-trimethyltryptammonium iodide according to claim 1.

9. A method of preventing or treating inflammation and/or pain comprising the step of:
administering to a subject in need thereof a composition according to claim 4.

10. A method of preventing or treating a psychological disorder comprising the step of:
administering to a subject in need thereof a composition according to claim 5.

11. A method of preventing or treating inflammation and/or pain comprising the step of:
administering to a subject in need thereof a composition according to claim 5.

12. A composition comprising crystalline N,N,N-trimethyltryptammonium iodide according to claim 2 and an excipient.

13. A composition comprising crystalline N,N,N-trimethyltryptammonium iodide according to claim 2 as a first component and a second component selected from at least one of (a) a serotonergic drug, (b) a purified psilocybin derivative, (c) a purified cannabinoid, (d) a purified terpene, (e) an adrenergic drug, (f) a dopaminergic drug, (g) a monoamine oxidase inhibitor, (h) a purified erinacine, and (i) a purified hericenone.

14. A method of preventing or treating a psychological disorder comprising the step of:
administering to a subject in need thereof a therapeutically effective amount of crystalline N,N,N-trimethyltryptammonium iodide according to claim 2.

15. A method of preventing or treating a psychological disorder comprising the step of:
administering to a subject in need thereof a composition according to claim 12.

16. A method of preventing or treating a psychological disorder comprising the step of:
administering to a subject in need thereof a composition according to claim 13.

17. A method of preventing or treating inflammation and/or pain comprising the step of:
administering to a subject in need thereof a therapeutically effective amount of crystalline N,N,N-trimethyltryptammonium iodide according to claim 2.

18. A method of preventing or treating inflammation and/or pain comprising the step of:
administering to a subject in need thereof a composition according to claim 12.

19. A method of preventing or treating inflammation and/or pain comprising the step of:
administering to a subject in need thereof a composition according to claim 13.

20. A composition comprising crystalline N,N,N-trimethyltryptammonium iodide according to claim 3 and an excipient.
